SEQUENCE LISTING (31 sequences)

SEQ ID NO: 1 - protein, 243 aa, Homo sapiens
DEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQCLAARLEALKENGGARLAEYHAKATEHLSTLSEKAKPALEDLRQGLLPVLESFKVSFLSALEEYTKKLNTQ

SEQ ID NO: 2 - DNA, 33 bp, Artificial Sequence
  chemically-synthesized primer
gttataaatttggagtgtgaaggttattgcgtg

SEQ ID NO: 3 - DNA, 33 bp, Artificial Sequence
  chemically-synthesized primer
cacgcaataaccttcacactccaaatttataac

SEQ ID NO: 4 - DNA, 32 bp, Artificial Sequence
  chemically-synthesized primer
gcggccgctatccattaacggatttgtgacag

SEQ ID NO: 5 - DNA, 31 bp, Artificial Sequence
  chemically-synthesized primer
gctgatgtcgacgccacatcggtttatctaa

SEQ ID NO: 6 - DNA, 50 bp, Artificial Sequence
  chemically-synthesized primer
cacgcaataaccttcacactccaaatttataacacgcattattctgctcc

SEQ ID NO: 7 - DNA, 55 bp, Artificial Sequence
  chemically-synthesized primer
gttataaatttggagtgtaaggttattgcgtgtaattaaaagccatacaagactg

SEQ ID NO: 8 - DNA, 38 bp, Artificial Sequence
  chemically-synthesized primer
gatgcagcggccgcgaggaaagtgctaaataataatca

SEQ ID NO: 9 - DNA, 27 bp, Artificial Sequence
  chemically-synthesized primer
gtagtagtcgacattgccagccccatc

SEQ ID NO: 10 - DNA, 51 bp, Artificial Sequence
  chemically-synthesized primer
cacgcaataaccttcacactccaaatttataaccattgttttttggactcc

SEQ ID NO: 11 - DNA, 50 bp, Artificial Sequence
  chemically-synthesized primer
gttataaatttggagtgtgaaggttattgcgtgtaatggcaatacgtggg

SEQ ID NO: 12 - DNA, 23 bp, Artificial Sequence
  chemically-synthesized primer
gtatagctggcaacgttctgttg

SEQ ID NO: 13 - DNA, 20 bp, Artificial Sequence
  chemically-synthesized primer
gatcaacgtgaacttcgtcc

SEQ ID NO: 14 - DNA, 21 bp, Artificial Sequence
  chemically-synthesized primer
agggcagggtcgttaaatagc

SEQ ID NO: 15 - DNA, 21 bp, Artificial Sequence
  chemically-synthesized primer
ttaatgcgccgctacagggcg

SEQ ID NO: 16 - DNA, 33 bp, Artificial Sequence; identical to SEQ ID NO: 2
  chemically-synthesized primer

SEQ ID NO: 17 - DNA, 33 bp, Artificial Sequence; identical to SEQ ID NO: 3
  chemically-synthesized primer

SEQ ID NO: 18 - DNA, 59 bp, Artificial Sequence
  chemically-synthesized primer
cacgcaataaccttcacactccaaatttataaccataatctatggtccttgttggtgaa

SEQ ID NO: 19 - DNA, 42 bp, Artificial Sequence
  chemically-synthesized primer
aaggaaaaaagcggccgctaaagtcgatttaccgcagccaga

SEQ ID NO: 20 - DNA, 56 bp, Artificial Sequence
  chemically-synthesized primer
gttataaatttggagtgtgaaggttattgcgtgtaatgctgtgaaatgccggatgc

SEQ ID NO: 21 - DNA, 37 bp, Artificial Sequence
  chemically-synthesized primer
cgcacgcatgtcgactgccaggagcgatctaacaaca

SEQ ID NO: 22 - DNA, 21 bp, Artificial Sequence
  chemically-synthesized primer
agggcagggtcgttaaatagc

SEQ ID NO: 23 - DNA, 21 bp, Artificial Sequence
  chemically-synthesized primer
ttaatgcgccgctacagggcg

SEQ ID NO: 24 - DNA, 16 bp, Artificial Sequence
  chemically-synthesized primer
gtaaaacgacggccag

SEQ ID NO: 25 - DNA, 17 bp, Artificial Sequence
  chemically-synthesized primer
caggaaacagctatgac

SEQ ID NO: 26 - DNA, 30 bp, Artificial Sequence
  chemically-synthesized primer
gcggccgccaaacagcgacaaaaagtgatg

SEQ ID NO: 27 - DNA, 59 bp, Artificial Sequence
  chemically-synthesized primer
cacgcaataaccttcacactccaaatttataaccccgcataaaagttctccattcaatc

SEQ ID NO: 28 - DNA, 55 bp, Artificial Sequence
  chemically-synthesized primer
gttataaatttggagtgtgaaggttattgcgtgggaataactagccatttcaatg

SEQ ID NO: 29 - DNA, 22 bp, Artificial Sequence
  chemically-synthesized primer
gtcgactgagcggcaatggcat

SEQ ID NO: 30 - DNA, 26 bp, Artificial Sequence
  chemically-synthesized primer
cccgcataaaagttctccattcaatc

SEQ ID NO: 31 - DNA, 22 bp, Artificial Sequence
  chemically-synthesized primer
cattgaaatggctagttattcc

(19) United States
(12) Patent Application Publication
(10) Pub. No.: US 2013/0029377 A1
(43) Pub. Date: Jan. 31, 2013

(54) RECOMBINANT APOA-1M FROM ENGINEERED BACTERIA

(71) Applicants: Maire H. CAPARON, Chesterfield, MO (US); Kevin J. RUST, Manchester, MO (US); Alan K. HUNTER, Damascus, MD (US); Joseph K. McLAUGHLIN, Ballwin, MO (US); Kristin E. THOMAS, Wildwood, MO (US); John T. HERBERG, Portage, MI (US); Robert E. SHELL, Charlotte, NC (US); Paul B. LANTER, Edwardsville, IL (US); Bruce F. BISHOP, Warrenton, MO (US); Robert L. DUFIELD, Windham, NH (US); Xing WANG, Wildwood, MO (US); Sa V. HO, Boston, MA (US)

(72) Inventors: Maire H. CAPARON, Chesterfield, MO (US); Kevin J. RUST, Manchester, MO (US); Alan K. HUNTER, Damascus, MD (US); Joseph K. McLAUGHLIN, Ballwin, MO (US); Kristin E. THOMAS, Wildwood, MO (US); John T. HERBERG, Portage, MI (US); Robert E. SHELL, Charlotte, NC (US); Paul B. LANTER, Edwardsville, IL (US); Bruce F. BISHOP, Warrenton, MO (US); Robert L. DUFIELD, Windham, NH (US); Xing WANG, Wildwood, MO (US); Sa V. HO, Boston, MA (US)

(73) Assignee: PFIZER INC., Groton, CT (US)

(21) Appl. No.: 13/557,855

(22) Filed: Jul. 25, 2012

Related U.S. Application Data
(60) Provisional application No. 61/511,439, filed on Jul. 25, 2011.
(60) Provisional application No. 61/538,406, filed on Sep. 23, 2011.

(51) Int. Cl. C12N 1/21; C12P 21/00

(52) U.S. Cl. 435/69.1; 435/252.33

(57) ABSTRACT

Apolipoprotein A-1 Milano (ApoA-1M), the protein component of a high-density lipoprotein (HDL) mimic with promising potential for reduction of atherosclerotic plaque, is produced on a large scale by expression in E. coli. Significant difficulty with clearance of host cell proteins (HCPs) was experienced in the original manufacturing process, despite lengthy purification. Analysis of purified protein solutions and intermediate process samples led to the identification of several major HCPs, and a bacterial protease causing the production of a truncated species of ApoA-1M co-purifying with the product. Deletions in these genes from the original host strain succeeded in substantially reducing the levels of HCPs without adversely affecting overall fermentation productivity, contributing to a much more efficient and robust new manufacturing process.

### CROSS-REFERENCE TO RELATED APPLICATIONS

The present application claims benefit of U.S. provisional application No. 61/511,439, filed Jul. 25, 2011, and U.S. provisional application No. 61/538,406, filed Sep. 23, 2011, the contents of both of which are incorporated herein by reference in their entireties.

### BACKGROUND OF THE INVENTION

Stringent product quality and safety requirements along with cost pressure represent significant challenges in the manufacture of recombinant therapeutic proteins. Recovery and purification of the protein product from the cells often constitute a large portion of the manufacturing process. Processing challenges include removal of process- and product-related impurities, often to extremely low levels. Separation of contaminants closely similar to the product can add considerable complexity to the manufacturing process and incur significant yield loss. A major group of process-related impurities comes from the host production system itself, and product-related impurities can arise during fermentation and/or be generated in downstream processing.

Apolipoprotein A 1 Milano (ApoA-1M) is a naturally occurring variant of ApoA-1, a protein present in human plasma in low concentration. ApoA-1 is the major protein component of high-density lipoprotein (HDL), which serves to maintain cholesterol homoeostasis and eliminate excess cholesterol (Donovan et al., 1987). The ApoA-1M variant was first identified in 40 individuals in Italy who had very low levels of high-density lipoprotein cholesterol (HDL-C), yet enjoyed apparent longevity and markedly less atherosclerosis than expected for their HDL-C levels (Gualandri et al, 1985; Sirtori et al, 2001). In a small, randomized controlled trial, treatment with five doses of intravenously administered ETC-216 (complex of ApoA-1M dimer and palmitoyl oleoyl phosphatidylcholine) at weekly intervals resulted in significant regression of coronary atherosclerosis as measured by intravascular ultrasound (Nissen et al, 2003).

ApoA-1M differs from wild-type ApoA-1 in that cysteine is substituted for arginine at position 173, permitting a disulfide-linked dimer to form. ApoA-1M with its ability to form a covalent dimer thus somehow appears to confer protection against cardiovascular disease to those that carry the mutated gene (Calabresi et al., 1994). Both ApoA-1 and ApoA-1M are composed of amphipathic alpha helices, leading to reversible self-association and the formation of dynamically interacting micellar-like structures in solution (Calabresi et al, 1994; Donovan et al 1987; Vitello and Scanu, 1976). ApoA-1 exists as a monomeric species only at very low protein concentration (<0.1 mg/mL); at 0.1 mg/mL, it is already mostly in dimer form, and at 1-2 mg/mL octamer is the predominant form (Donovan et al, 1987). The amino acid sequence of ApoA-1M is shown in FIG. 1. ApoA-1M monomer consists of 243 amino acids with a molecular weight of around 28 kDa (56 kDa for dimer) and isoelectric point (pI) of 5.1-5.3.

The original manufacturing process for ApoA-1M dimer has been described (Hunter et al, 2008a). It is quite complex, comprising E. coli fermentation with ApoA-1M expressed in the periplasm and a downstream purification that consists of 15 steps, including 5 chromatography columns (3 ion-exchange and 2 hydrophobic interactions). Due to its characteristics, ApoA-1M tends to bind to many impurities, such as host cell proteins, lipids, and endotoxins, rendering its purification to the standard expected for biotherapeutics particularly difficult. High urea concentration is used throughout the process to minimize both ApoA-1M self-association and its association with certain impurities (Hunter et al, 2007). Despite the rather lengthy purification process, several initial batches from a manufacturing campaign failed the stringent Host Cell Protein (HCP) requirement of ≦10 ng/mg protein (10 ppm) in the purified protein product, even though all other specified product quality characteristics were very good. Only with a rather empirical modification of a critical chromatography step, a hydrophobic interaction column (HIC) combined with very narrow protein loading ranges for the other chromatography columns was the HCP level sufficiently reduced. Details on developmental activities and large-scale operation of the HIC column with respect to HCP clearance have been described in (Hunter et al, 2008b).

Even though the original manufacturing process could produce purified proteins that met product specifications for clinical trials, there were many potential issues. First, the complex purification scheme of the process and the very narrow operating ranges for the columns were a major source of concern. For commercial manufacturing, significant process improvement would be required for process robustness as well as for cost reduction; the latter would typically involve reducing the number of major processing steps such as chromatography columns. Second, analysis of the residual HCP's in the purified protein batches by Western blot shows that even those that met the HCP specification still contained several distinct bands of HCPs, which apparently persisted through five chromatography columns (Caparon et al., Biotechnology and Bioengeneering vol 105, 239-249, 2010, incorporated herein by reference for all purposes). The significance of these impurities with respect to process robustness and especially safety was not known. Another issue was the detection of a major truncated version of ApoA-1M at noticeable levels in the purified protein batches. The truncated version, called K238/K239, is five amino acid residues shorter from the C terminus of ApoA-1M, indicating that it was probably formed by either chemical or proteolytic cleavage between the two lysines at positions 238 and 239 (see FIG. 1). While product heterogeneity is not unusual for biotherapeutics, it is always best to try to keep it to a minimum if possible. Because of the close similarity between ApoA-1M and K238/K239, removal of the latter by purification would be very difficult and costly. There is a need, therefore, for an effective, integrated approach in which these particular purification challenges are addressed.

### SUMMARY OF THE INVENTION

The present invention provides E. coli cell lines modified to prevent expression of selected genes, E. coli cell lines modified to prevent expression of selected genes and engineered to express a polynucleotide encoding apolipoprotein A-1 Milano (“ApoA-1M”), pharmaceutical compositions comprising ApoA-1M, ApoA-1M:lipid complexes, pharmaceutical compositions comprising ApoA-1M:lipid complexes, ApoA-1M:phospholipid complexes, pharmaceutical compositions comprising ApoA-1M:phospholipid complexes, methods of producing and purifying ApoA-1M, and methods of treatment by administering a pharmaceutical composition comprising ApoA-1M or one of the complexes disclosed herein.

In one embodiment, the invention can be described as an isolated E. coli cell line wherein the cell line has been modified to prevent expression of one or more of the oppA gene, the dppA gene, the malE gene, and the ompT gene, or any combination thereof. Expression of the cited genes can be prevented by various means known in the art. Preferably, expression is blocked by making chromosomal deletions that prevent expression of the gene. Such deletions can include deletions of the entire coding region of a gene, or any part of the coding region or control regions that result in a lack of expression of the entire coding region. In particular, the result of such a deletion is the inability of the cell line to produce the protein encoded by the gene.

E. coli cell lines modified to prevent expression of one or more of the oppA gene, the dppA gene, the malE gene and the ompT gene, or any combination thereof, included within the scope of the invention are cell lines modified to prevent expression of the oppA gene; the dppA gene; the malE gene; the ompT gene; the oppA gene and the dppA gene; the oppA gene and the malE gene; the oppA gene and the ompT gene; the dppA gene and the malE gene; the dppA gene and the ompT gene; the malE gene and the ompT gene; the oppA gene, the dppA gene and the malE gene; the dppA gene, the malE gene and the ompT gene; the oppA gene, the dppA gene and the ompT gene; the oppA gene, the malE gene and the ompT gene; and the oppA gene, the dppA gene, the malE gene, and the ompT gene.

E. coli cell lines having one or more chromosomal deletions that prevent expression of a gene included within the scope of the invention are cell lines having a deletion of the oppA gene, the dppA gene, the malE gene, or the ompT gene or any combination thereof, including cell lines with a deletion in the oppA gene; a deletion in the dppA gene; a deletion in the malE gene; a deletion in the ompT gene; a deletion in the oppA gene and the dppA gene; a deletion in the oppA gene and the malE gene; a deletion in the oppA gene and the ompT gene; a deletion in the dppA gene and the malE gene; a deletion in the dppA gene and the ompT gene; a deletion in the malE gene and the ompT gene; a deletion in the oppA gene, the dppA gene and the malE gene; a deletion in the dppA gene, the malE gene and the ompT gene; a deletion in the oppA gene, the dppA gene and the ompT gene; a deletion in the oppA gene, the malE gene and the ompT gene; and a deletion in the oppA gene, the dppA gene, the malE gene, and the ompT gene.

In another embodiment the present invention includes each of the cell lines set forth herein, wherein the cell line has been further engineered to express a polynucleotide encoding a human Apolipoprotein A-1 (ApoA-1), such as human Apolipoprotein A-1 Milano (“ApoA-1 Milano” or “ApoA-1M”) as described herein, the amino acid sequence of which is shown in FIG. 1. The apolipoprotein can be expressed from a vector or plasmid comprising a polynucleotide encoding the protein in any of the cell lines disclosed herein; in particular the protein can be expressed from a plasmid. A suitable host cell for the production of the apolipoprotein, such as ApoA-1M as described herein, is an E. coli BC50 cell.

In a further embodiment the present invention is directed to a method of producing a protein comprising providing a culture of any of the E. coli cell lines set forth herein, i.e., wherein the cell line comprises a chromosomal deletion in one or more of the oppA gene, the dppA gene, the malE gene, and the ompT gene or any combination thereof, and further wherein the cell line comprises a polynucleotide encoding the protein; growing the cell culture under conditions that allow expression of the polynucleotide encoding the protein, and collecting the protein from the cell culture. In certain embodiments, the protein has a molecular weight of from 45 to 75 kDa and an isoelectric point of from pH 4-6. In preferred embodiments, the protein is a human apolipoprotein A-1 or ApoA-1 Milano as set forth in SEQ ID NO:1.

Practice of the method can include cultures in which the cell line includes a deletion in the oppA gene; the dppA gene; the malE gene; the oppA gene and the dppA gene; the oppA gene, the dppA gene and the malE gene; the ompT gene; or the oppA gene, the dppA gene, the malE gene, and the ompT gene. The method may also be practiced using any of the cell lines disclosed herein.

A further embodiment of the invention is a method of producing a purified recombinant human apolipoprotein A-1 Milano (“rApoA-1M”), the method comprising providing an E. coli cell line, wherein the cell line has been engineered to express a polynucleotide encoding human apolipoprotein A-1 Milano and contains a chromosomal deletion in one or more of the oppA gene, the dppA gene, the malE gene and the ompT gene, or any combination thereof, including any of the cell lines set forth herein; growing the cells under conditions effective to express the polynucleotide encoding the human apolipoprotein A-1 Milano; heat extraction of a protein containing fraction from the cell culture media; reduction of the protein containing fraction by treatment with a thiol reductant; contacting the reduced protein containing fraction with a reversed phase capture column; contacting the protein containing fraction with an anion exchange column; contacting the protein containing fraction with a hydrophobic interaction phenyl column; contacting the protein containing fraction with a Cu(II) oxidant; and contacting the protein containing fraction with an anion exchange Q column.

In a further aspect of the invention, the invention includes purified recombinant human apolipoprotein A-1 Milano (“rApoA-1M”) that contains less than 10 ng, less than 5 ng, or less than 3 ng of host cell protein per mg of protein. In another aspect, the rApoA-1M can be complexed with a lipid, such as a phospholipid. In one embodiment, the phospholipid is POPC. The rApoA-1M:lipid complex can be contained in a pharmaceutically acceptable carrier.

The present invention further includes methods of producing a pharmaceutical composition for the treatment of cardiovascular disease, atherosclerosis or acute coronary syndromes that comprises isolating rApoA-1M from a culture of E. coli engineered to express a polynucleotide encoding apolipoprotein A-1 Milano, wherein the E. coli comprise one or more deletions in the oppA gene, the dppA gene, the malE gene and the ompT gene, or any combination thereof, and includes any of the cell lines set forth herein; contacting the protein fraction from the culture with a single sorbent capture column and eluting the protein from the column to produce a capture pool; contacting the capture pool with a single ion exchange column and eluting the protein from the column to obtain purified rApoA-1M; and preparing a composition comprising the purified protein in a pharmaceutically acceptable carrier to obtain a pharmaceutical composition. The method can further include adding a lipid, such as a phospholipid, including POPC, to the purified protein to obtain a pharmaceutically effective apolipoprotein A-1 Milano:lipid complex, including an apolipoprotein A-1 Milano:POPC complex.

Another aspect of the invention is methods of treating a cardiovascular disease, a vascular disorder, an ischemic disorder, atherosclerosis or an acute coronary syndrome comprising administering to a subject in need thereof, a composition, including a pharmaceutically acceptable composition, comprising recombinant ApoA-1M, wherein the composition comprises less than 3 ng host cell protein per mg of recombinant ApoA-1M. The disclosed rApoA-1M is effective, when administered to a mammal at a dosage of 300 mg/kg results in an AUC₀₋₂₄ of greater than 100,000 μg*h/ml.

An aspect of the invention can also be described as a pharmaceutical composition comprising recombinant apolipoprotein A-1 Milano (rApoA-1M) complexed with POPC and contained in a pharmaceutical carrier, wherein the composition comprises less than 3 ng host cell proteins per mg of rApoA-1M. In certain embodiments the composition is free of bacterial OppA protein, the bacterial DppA protein, the bacterial MalE protein or the bacterial OmpT protein.

The recombinant apolipoprotein A-1 Milano is purified from an E. coli culture by the steps as shown in the right hand column of FIG. 7 and exhibits significantly improved pharmacokinetic properties such as Cmax and Area Under the Curve (AUC) than ApoA-1M produced from E. coli cells that do not include the described chromosomal deletions, and that is purified by the steps shown in the left hand column of FIG. 7.

As used herein, the term “substance” includes but is not limited to one or more active-ingredient-containing substances wherein the active ingredient may be a biologic agent such as a protein, peptide, vaccine, or an active pharmaceutical ingredient (“API”), for example a pharmaceutical drug such as a prescription drug, generic drug, or over-the-counter pharmaceutical, nutraceutical or homeopathic product. The substance may be in an aqueous, gel, powder, solution, emulsion, crystals or suspension form. As used here, the term “substance” is interchangeable with the terms “drug,” “drug product,” “medication,” “liquid,” “biologic,” “active ingredient” or “API.”

As used herein, an “active ingredient” or API is any component intended to furnish pharmacological activity or other direct effect in the diagnosis, cure, mitigation, treatment, or prevention of disease, or to affect the structure or any function of the body of humans or other animals.

As used herein, the term “unit dosage form” is interchangeable with the terms “bottle,” “vial,” “unit-dose,” “dosage form,” “unit-dose vial,” “blister,” “dosage blister,” “ampoule” or “container.”

As used herein, the term “treatment” or “treating” includes treating an active condition, or preventing or inhibiting a condition or disorder in a subject that is at risk of developing such a condition or disorder. Treatment is not limited to curing a disease or disorder, or reaching a certain end point, but includes the administration of the treatment regardless of outcome.

Throughout this disclosure, unless the context dictates otherwise, the word “comprise” or variations such as “comprises” or “comprising” is understood to mean “includes, but is not limited to” such that other elements that are not explicitly mentioned may also be included. Further, unless the context dictates otherwise, use of the term “a” may mean a singular object or element, or it may mean a plurality, or one or more of such objects or elements. In addition, the use of “or” herein means “and/or” unless specifically stated otherwise. Also, terms such as “element” or “component” encompass both elements and components comprising one unit and elements or components that comprise more than one unit, unless specifically stated otherwise.

It is to be understood that both the foregoing general description and the following detailed description are exemplary and explanatory only, and are not restrictive of the inventions, as claimed. The section headings used herein are for organizational purposes only and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

### DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 is the amino acid sequence of ApoA-1M monomer (SEQ ID NO:1).

FIG. 2 shows the relative locations of the oligonucleotides on the E. coli chromosome used to generate the ompT deletion construct and its subsequent subcloning into the pKO3 plasmid.

FIGS. 3A-1-3 and 3B-1-3 are the high pressure liquid chromatography (HPLC) analyses of purified ApoA-1M samples. FIG. 3A shows the truncated species AA 1-238 identified by mass spectrometry (MS) as a major fragment co-eluting with the intact ApoA-1M from the triple knockout strain (FIG. 3A-3 shows K238/239 Truncation Coeluting with ApoA 1-M). FIG. 3B taken from the quadruple knockout strain shows the absence of the AA 1-238 fragment (FIG. 3B-3 shows K238/239 Truncation Not Observed).

FIG. 4 is a map of the pKO3 vector.

FIG. 5 is a schematic of the PCR strategy for construction of minigene inserts used to knock out the four host cell protein genes in the host E. coli cells.

FIG. 6 is a schematic of the ompT gene region with location of the oligos used in construction of the minigene construct.

FIG. 7 is a comparison of the downstream purification steps in the prior process (left side) and new process disclosed herein (right side).

### DETAILED DESCRIPTION OF THE INVENTION

The present disclosure arises at least in part from a need for improved methods of production for biotherapeutic products and for improved biotherapeutic agents produced by the novel methods disclosed herein. A problem in the production of biotherapeutics in large scale fermentation procedures is the presence of host cell proteins (HCPs) in the purified product. This is particularly difficult when the HCPs are similar to the desired product in physical and/or chemical characteristics. In the example of ApoA-1M, production in E. coli results in a number of HCPs that are similar to the product in molecular weight and net charge. The purification of the recombinant product, therefore, requires a long and complex series of steps to achieve even minimal purity standards.

The present disclosure is drawn to a molecular biology based solution to the problem of purification of recombinant biotherapeutics. The inventors have demonstrated herein the production of modified and improved host strains with multiple gene deletions that are able, nonetheless, to exhibit robust cell growth and protein expression of the desired product. The fermentation product is thus easier to purify with several steps in the conventional processes no longer necessary to achieve a greater degree of purification than could be demonstrated with a product of the conventional host cells. The product is also shown herein to be surprisingly superior in bioavailability as shown in Example 4, below. The success of this approach also highlights a key advantage of protein expression in E. coli in that it is possible to delete many genes without adversely affecting cell growth and the overall complement of proteins needed for effective expression of the desired product.

### Detection and Identification of Co-Purified Host Cell Proteins

HCPs that were difficult to remove from purified ApoA-1M in spite of a very complex purification process represented a particularly challenging purification problem and substantial process improvement was required. Several distinct HCP bands were still detected in the purified protein batches produced by the original process (Caparon et al., 2010). Finding out what these were or at least determining their physicochemical properties greatly facilitated the process improvement effort.

The initial investigation started with the purified protein batches available from production, and focused on the prominent HCP bands. Multiple analytical and biochemical methods were employed including 1-dimensional (1D) and 2-dimensional (2D) gel electrophoresis, Western blots, protein deglycosylation, mass spectrometry (MS), Edman degradation, and a sandwich ELISA. This particular ELISA was developed using anti-E. coli HCP antibodies and was based on the glycosylation property of the major HCPs identified.

Two-dimensional Western blot analysis of the purified ApoA-1M samples detected two major HCPs along with 6-7 minor HCP species. These two major HCPs have molecular weights around 60 kDa, and exhibit multiple forms on the 2D gel of a purified protein sample. Both the molecular weight and isoelectric point of the HCPs are thus quite close to those of ApoA-1M dimer (56 kDa, pI: 5.1-5.3), which may explain why they co-purified with ApoA-1M.

Purified protein samples, however, contained too little of the HCP contaminants for detailed analysis that could lead to their identification. Critical to the characterization effort was the availability of intermediate process samples that contained relatively high levels of three contaminating HCPs. The Western blot result obtained with purified protein samples was used as a reference to locate the major HCPs detected in an intermediate process sample on a Sypro Ruby stained gel. These were confirmed to be HCPs by Western blot analysis of the same gel. Two-dimensional gel electrophoresis was then used to separate and isolate the major HCP spots for identification by MS sequencing. The MS data were processed using the Protein Lynx software package (Micromass, Beverly, Mass.), MASCOT (Matrix Science, London, UK) and the SwissProt database, selected for E. coli proteins.

From the above investigation, two HCPs were identified as E. coli periplasmic proteins involved in transport of peptides. These were a dipeptide binding protein (DppA) and an oligopeptide binding protein (OppA), that are encoded by the genes dppA and oppA, respectively. Their locations on the E. coli chromosome are 79.84 min for dppA and 28.00 min for oppA. Both genes are 1.6 kb in size, resulting in mature proteins of 60 kDa, which is quite close to the 56 kDa for ApoA-1M dimer. Additionally, the net charges of DppA and OppA are both similar to that of ApoA-1M. DppA and OppA are among the largest and most abundant proteins in the periplasm of some E. coli strains (Abouhamad and Manson, 1994; Abouhamad et al, 1991; Guyer et al., 1985; Hiles and Higgins, 1986; Olson et al., 1991). OppA protein is distinguished from other periplasmic binding proteins by its broad specificity (Doeven et al., 2004; Tame et al., 1994). Both OppA and DppA were identified as glycoproteins through glycanase treatment, Western blot analysis, and the successful development of a glycol-ELISA based on their glycosylation.

A third major co-purifying HCP was identified as maltose-binding periplasmic protein (MalE). This protein is encoded by the malE gene, located at the 91.46 min position on the E. coli chromosome. The malE gene is ˜1.2 kb in length and codes for a protein with an apparent molecular weight of 43 kDa. Like DppA, MalE is localized to the periplasmic space, where it is engaged in the high-affinity active transport of maltose into the cells, and is responsible for chemotaxis toward malto-oligosaccharides (Duplay et al., 1984; Spurlino et al., 1991).

Although not identified as a co-purifying HCP per se, a bacterial protease was suspected as causing the cleavage between the two lysine residues at amino acids 238 and 239 of ApoA-1M to form the K238/K239 truncated species (see FIG. 1). In the original production process, this species made up from 2% to 9% of total ApoA-1M in the purified protein batches. Because of its affinity for paired basic residues in unfolded or expressed proteins, and because it is localized to the periplasmic space, the outer membrane protease 3B (Protease 7 precursor, or OmpT; McCarter et al., 2004) was thought to be a likely candidate causing this specific truncation of ApoA-1M. OmpT is a 35.5 kDa protease from the peptidase A26 family (Grodberg et al., 1988; Kramer et al., 2000) and is encoded by the gene ompT, located at 12.59 min on the E. coli chromosome.

### Genetic Approach for Removal of Problematic HCPs

With the genes encoding the proteins that caused complications in downstream purification identified, one promising approach was to delete them from the E. coli host strain, thereby removing the problematic impurities from the starting material and simplifying the production process. The inventors decided to explore the effects of deleting the four identified genes from the host strain in a step-wise manner; first the pair oppA and dppA, followed with malE for further HCP reduction, then finally ompT for potential minimization of the undesirable truncation.

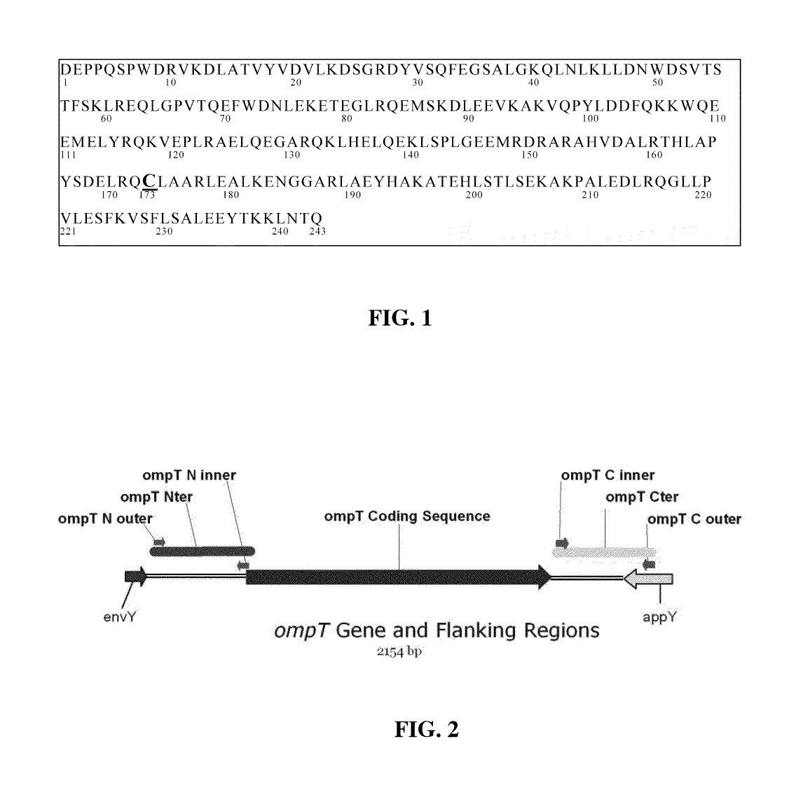

The deletion method used has been described previously (Link et al, 1997). The generation of individual deletion constructs is similar for each gene. FIG. 2 shows the relative locations of the oligonucleotides on the E. coli chromosome used to generate the ompT deletion construct and its subsequent subcloning into the pKO3 plasmid. The gene deletion procedure was first performed on the BC50 parent strain using dppA, oppA and malE deletion plasmids sequentially. The ΔdppAΔoppAΔmalE deletions were confirmed by PCR analysis. Competent BC50 ΔdppAΔoppAΔmalE cells were then transformed with pKO3-ompT knockout plasmid and taken through the deletion procedure as described (Link et al., 1997, incorporated herein by reference for all purposes). In order to demonstrate that all of the desired deletions were retained in the quadruple knockout strain, PCR analysis was performed on genomic DNA from the cells. Outer primer pairs for each of the target genes were used to amplify the genomic area associated with the genes, in comparison with the progenitor strain BC50. In each case the amplified section of the genome is truncated in the DNA of the quadruple knockout strain relative to the BC50 control. This strain was assigned the name GB004, and later used to manufacture the master and working cell banks.

### Effects of Gene Deletions on Process Performance

### Reduction of Overall HCP Load

Comparative 2D HCP Western blots confirmed the removal of the two major HCPs. The BC50 parental cells clearly showed the presence of spots identified as the dppA and oppA gene products. In contrast, the gel image of a comparable sample from the ΔdppAΔoppA cells showed no protein spots at the same positions in the gel, indicating the absence of the targeted proteins (Caparon et al., 2010). Thus, deletion of the dppA and oppA genes succeeded in the elimination of the two major host cell protein contaminants.

Deletion of malE from the double knockout strain (ΔdppAΔoppA) had a surprisingly large impact on the HCP load after the elimination of OppA and DppA. Table 1 shows the total amounts of HCP after two key process steps, the first chromatography column (the Capture column) and the subsequent ion-exchange (DEAE) column. After the Capture column, the malE deletion resulted in a ˜35-fold reduction in the amount of HCP. After the DEAE column the malE deletion resulted in a ˜25-fold reduction in HCP level, to a value of <3 ng/mg protein. With this triple knockout host strain, not only were the two co-purified OppA and DppA proteins removed, the HCP requirement of 10 ng/mg protein could be achieved after only two chromatography steps. This was a remarkable improvement over the product obtained from the original strain and manufacturing process.

TABLE 1
Effects of HCP deletions on process performance: impact of
malE deletion on HCP load and effect of ompT deletion on
levels of truncated ApoA-1M

| | | HCP level |
|---|---|---|
| Process Sample | E. coli strain | (ng HCP/mg ApoA-1M) |
| Capture column pool | ΔdppAΔoppA | 22,800 |
| | ΔdppAΔoppAΔmalE | 660 |
| DEAE column pool | ΔdppAΔoppA | 75 |
| | ΔdppAΔoppAΔmalE | <3 |
| | ApoA-1M K238/K239 truncated species | |
| | (% total ApoA-1M) | |
| | ΔdppAΔoppAΔmalE | ΔdppAΔoppAΔmalEΔompT |
| Capture pool | 1.9 | 0.1 |
| DEAE pool | 1.3 | 0.2 |
| DEAE post-peak pool | 7 | 1.4 |
| Selected DEAE post- | 14.2 | 1.6 |
| peak fraction | | |

### Reduction of C-Terminal Truncated Species

The impact of the ompT deletion in the triple knockout host strain was first checked with samples obtained after the Capture column step and the DEAE chromatography step, respectively. The results are shown in Table 1 for the “3 KO” strain (ΔdppAΔoppAΔmalE) and the GB004 quadruple knockout strain (ΔdppAΔoppAΔmalEΔompT).

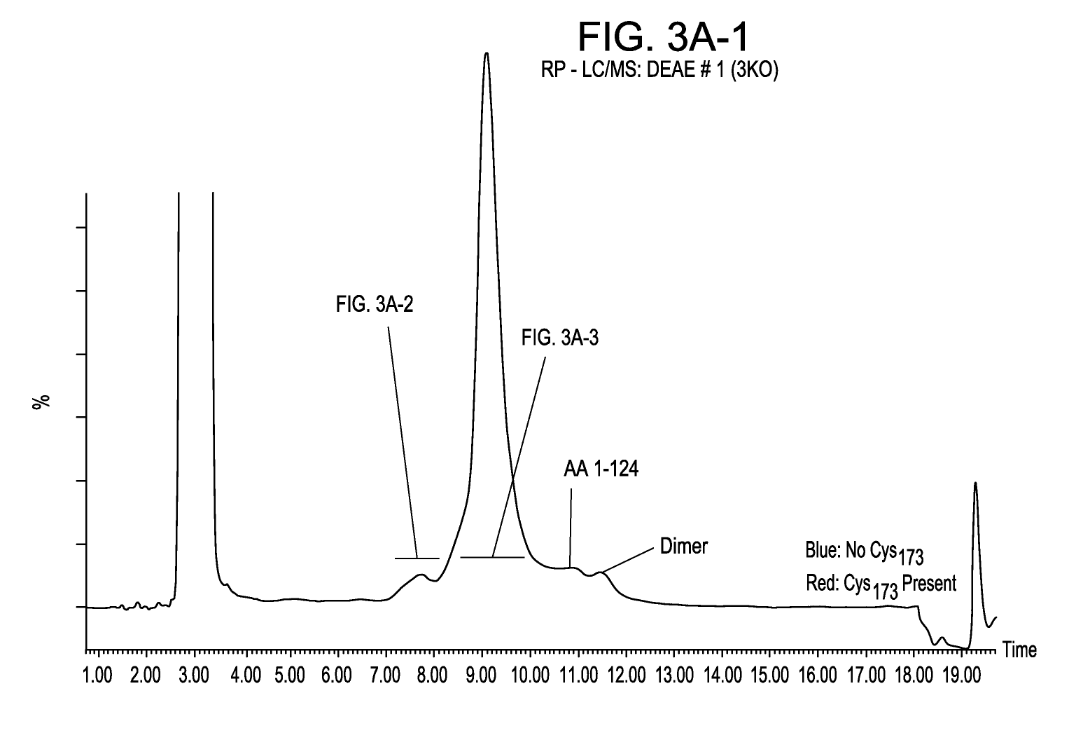

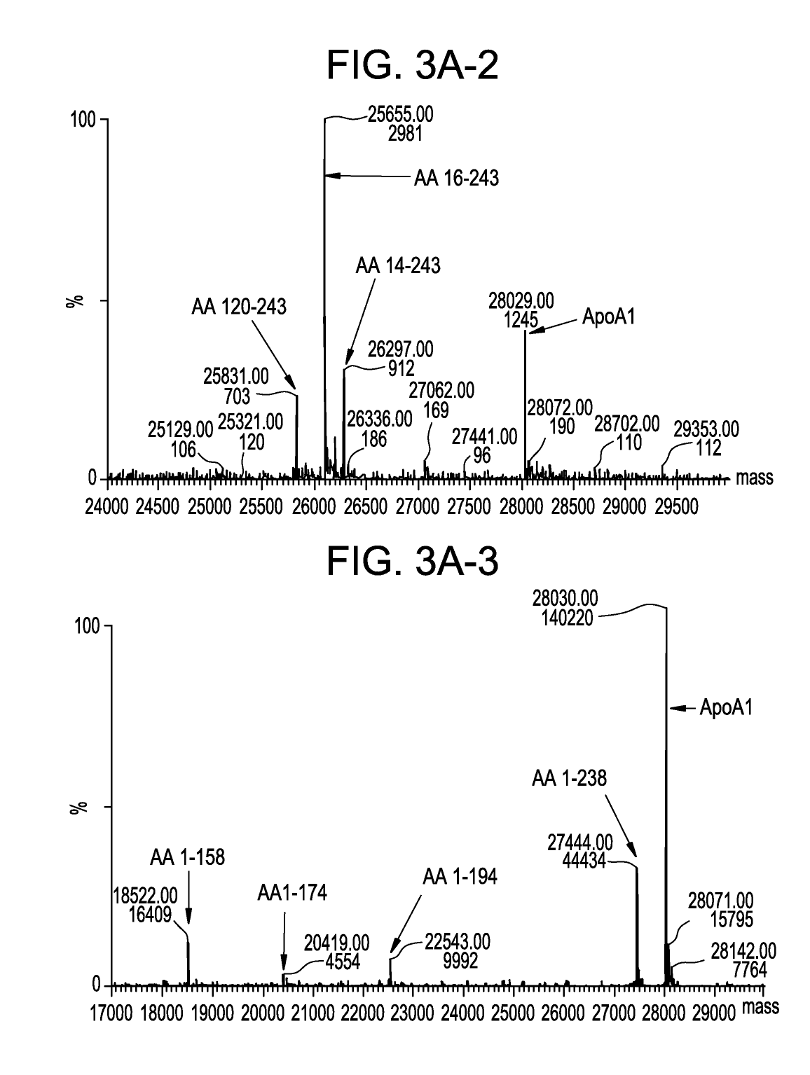

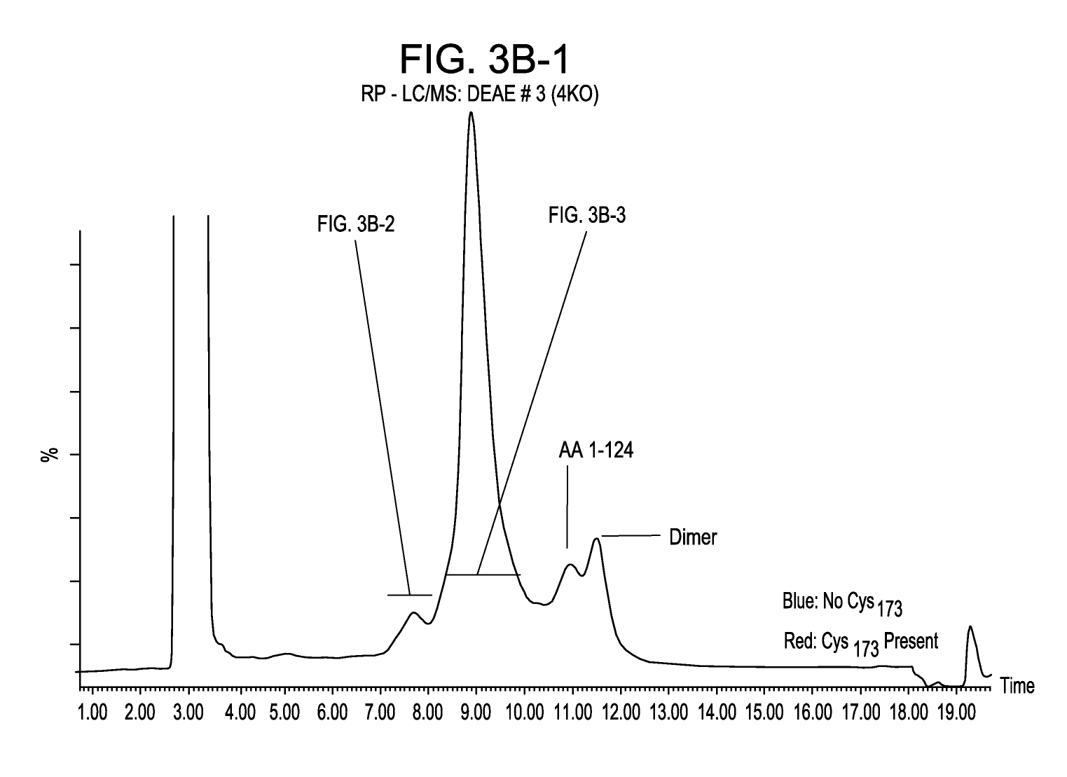

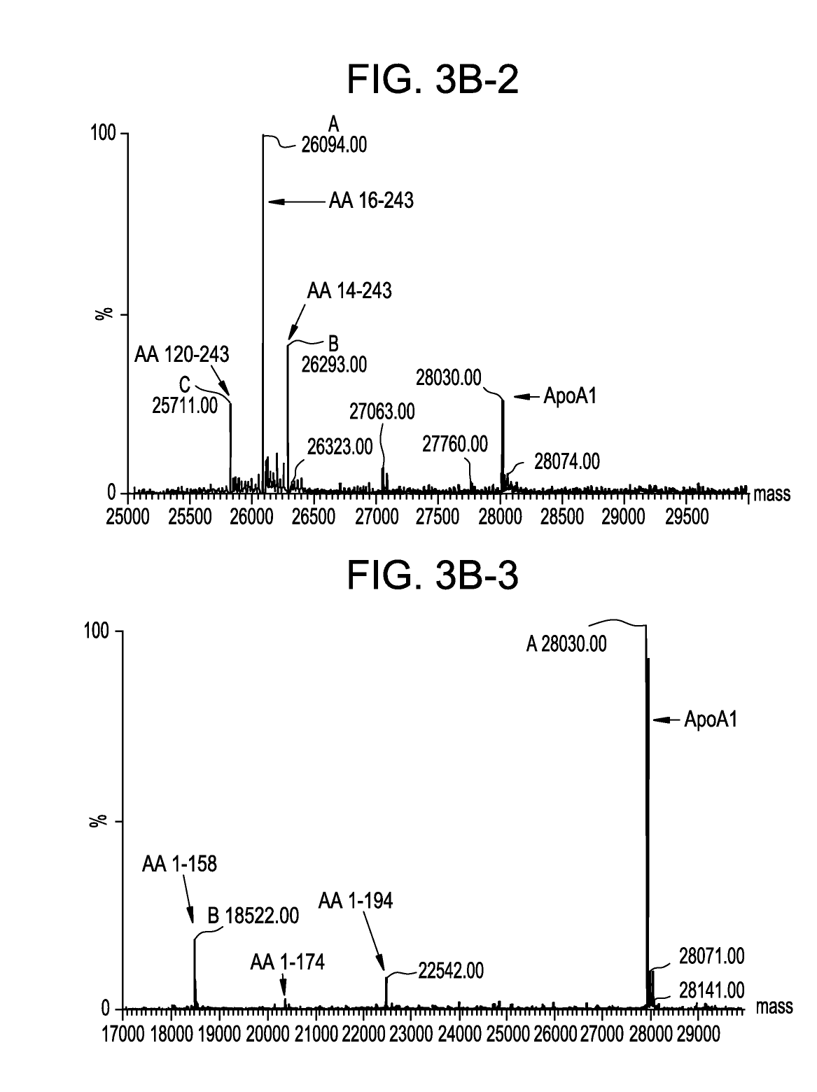

Each of the two process steps examined shows a marked (˜5- to 20-fold) reduction in the amount of truncated ApoA-1M in the GB004 strain compared with the 3K0 strain. These results showed that the OmpT protease was responsible for the K238/K239 truncation of ApoA-1M. Subsequent analysis of purified ApoA-1M samples with HPLC-MS confirmed the absence of the K238/K239 truncated species in protein solutions prepared with the quadruple knockout strain (FIG. 3B), whereas it was still a predominant variant in the triple knockout preparations (FIG. 3A). Given the similarity of the truncated species to the full-length ApoA-1M, removal of the truncated species from the final product would have been very daunting. Elimination of the source of the truncation (the OmpT protease) thus represents a highly effective solution to this challenging purification problem.

### Fermentation Process Performance

While the targeted gene deletions succeeded in greatly reducing HCP loads and generation of the truncated product species, an important issue with respect to the commercial feasibility of the process was the potential adverse impact of genetic modifications on growth and productivity of the host strain. Fermentation characteristics of the genetically modified host strain were therefore carefully checked, first in shake flasks then in 10-L fermenters, after each successive deletion of the four genes.

Like the double and triple knockout strains, the final quadruple knockout strain exhibited similar growth characteristics to the original host strain. This enabled conventional process optimization to be carried out. Key process parameters investigated in the optimization study included media pH and compositions, process temperature, glucose feed regime, induction time and cell density, and harvest time, using the design of experiment (DOE) approach. From this study, the fermentation process, optimized with the new host strain, raised the ApoA-1M titer to almost 5 g/L at harvest, compared to 3.2 g/L with the original host strain. This was achieved utilizing a combination of improved glucose feeding protocol and raising both temperature shift and cell density at induction. The higher titer appears to result from both higher cell density and higher cell productivity. The harvest dry cell weight for the improved fermentation process with the new host strain was about 66 g/L compared to 54 g/L with the original host strain. The average cell specific productivity of the original host strain was calculated to be about 0.06 g of ApoA-1M per gram of dry cell weight biomass, compared to about 0.08 g with the new strain. A summary of the key fermentation parameters for the two processes is listed in Table 2.

TABLE 2
Fermentation comparison between the original host strain and
the quadruple knockout strain.

| | E. coli strain | |
|---|---|---|
| Parameter | Original^{a} | Quadruple KO^{b} |
| Induction density (OD 600 nm) | 99 ± 5 | 133 ± 9 |
| Harvest density (OD 600 nm) | 134 ± 2 | 171 ± 8 |
| Dry cell wt at harvest (g/L) | 54 ± 1 | 66 ± 5 |
| ApoA-1M titer at harvest (g/L) | 3.2 ± 0.4 | 4.9 ± 0.1 |
| Specific productivity | 0.059 ± 0.008 | 0.076 ± 0.006 |
| (g ApoA-1M/g dry cell wt biomass) | | |

^{a}Average of 3 runs
^{b}Average of 5 runs

Deletion of the four identified genes did not adversely affect either growth or productivity of the cells and also allowed traditional optimization of the fermentation step to contribute to the improvement of the overall manufacturing process.

### Materials and Methods

All chemicals used such as salts, buffers, polymers, and surfactants were either purchased from Sigma or from suppliers as identified. Purified protein solutions and in-process samples were obtained directly from pilot plant and manufacturing operations.

### Analytical Methods

### HPLC and HPLC/MS Methods

Concentrations of Apo A-1M monomer, dimer, and aggregates were measured using size exclusion high-performance liquid chromatography (SEC HPLC) in the presence of SDS. An Agilent 1100 HPLC (Santa Clara, Calif.) was used with a TSKgel G3000SWXL (300 mm×7.8 mm) size-exclusion column obtained from Tosoh Bioscience (Montgomeryville, Pa.). The amounts of ApoA-1M monomer and many of its various chemically modified forms such as deaminated and carbamylated species were measured with an anion exchange HPLC method. The analysis was performed using a ProPac SAX-10 column obtained from Dionex (Sunnyvale, Calif.) with an Agilent 1100 HPLC.

The identification of various chemically modified forms of ApoA-1M monomer was accomplished by isolating each species from the anion-exchange HPLC method described above, followed by trypsin digestion and analysis by reversed phase HPLC electrospray ionization mass spectrometry (RP-HPLC/ESI-MS). Analysis of each trypsin digest was carried out with an Agilent 100 HPLC coupled to a Waters Q-TOF Micro mass spectrometer (Milford, Mass.). Each digest was injected onto a Zorbax 300 SB C18 (2.1 mm×100 mm) column obtained from Agilent maintained at 30° C. Peptides were identified utilizing BioLynx® and compared to the native peptide masses of ApoA-1M.

### Enzyme-Linked Immunosorbent Assay (ELISA) and Electrophoretic Methods

The concentration of E. coli HCP in ApoA-1M containing samples was determined using a process-specific ELISA method. For 1D gel electrophoresis, Bio-Rad (Hercules, Calif.) pre-cast 18-well gels with loading capacity of 30 μl were used. Two-dimensional gel electrophoresis was carried out following manufacturer's instruction (Bio-Rad). For the first dimension IEF (isoelectric focusing), wide range pH IPG (immobilized pH gradients) strips (pH 3-10) were used. All the reagents were obtained from Bio-Rad and prepared according to manufacturer's instruction. Gels were stained with either SYPRO Ruby or Silver reagents, purchased from Bio-Rad. SYPRO Ruby staining was done overnight at room temperature with shaking and images taken with a Hitachi CCD camera. Silver staining was done according to manufacturer's instruction, and the image was scanned with a Bio-Rad Scanner.

For Western blot analysis, proteins were first separated in SDS-PAGE, then transferred to nitrocellulose membrane (Bio-Rad) with TrisGly transfer buffer from Life Technologies (Carlsbad, Calif.). Primary antibodies from Cygnus Technologies (anti-E. coli HCP) were used; the secondary antibody was Donkey anti-Goat IgG (H&L) with IRDye800 conjugate. The membrane was scanned with the Odyssey Image system in the 800 nm channel.

For the MS identification of the major HCPs, 17-cm immobilized pH gradient (IPG) strips (non-linear, pH 3-10) were used with total protein loading of 400 μg/gel. After isoelectric focusing, the acidic part of the strip (about 12 cm) was cut and retained for second dimension separation. The spots containing the proteins of interest were cut from the gel and subjected to a trypsin digestion process for MS identification.

### Genetic Methods

### Strains and Culture Conditions

E. coli strain BC50 served as the progenitor strain for genetic modifications. BC50 is a K12 derivative with the genotype ayl ara T4^{R} and was the original strain used for ApoA-1M production. Bacterial stocks were cultured and maintained on standard LB broth or agar, obtained from Teknova (Hollister, Calif.). Media for culture was supplemented with antibiotic or sucrose as called for in the selection procedure. Cultures were typically maintained at 37° C., but shifted to 30°, 39°, or 42° C. as required.

### Primer Selection and Gene Deletion Procedure

The PCR primer pairs used to make deletion constructs were selected by the method of Link et al. (1997) for use with plasmid pKO3. The oligos were purchased from Sigma/Genosys (The Woodlands, Tex.). DNA sequences corresponding to the N-terminal and C-terminal flanking regions of the targeted gene were individually generated by PCR from an E. coli K12-derived genomic DNA template using the method of Link et al. (1997). Candidate knockouts were analyzed by performing colony PCR using oligos representing the flanking regions of the gene. Putative knockouts identified in this manner were grown overnight in a small volume of LB and used to generate genomic DNA. Purified genomic DNA was analyzed by PCR reactions utilizing the flanking primer set and a set of internal primers targeted to the gene of interest. Results of the PCR reactions were analyzed on 1.2% agarose E-Gels (Life Technologies).

### Fermentation Protocol

The fermentation process consisted of inoculum preparation, seed fermentation, and production fermentation. Typically, a vial was thawed and expanded in a shake flask to prepare the inoculum for the seed fermenter, which was then used to inoculate the production fermenter. The culture was grown at 30° C. during the pre-induction growth phase with dissolved oxygen maintained at ≧30%. Glucose concentration was monitored throughout the fermentation with an YSI 2700 glucose analyzer. At the depletion of the initial batched glucose, a glucose methionine feed solution was initiated and maintained until the end of the fermentation. Once the culture reached a target cell density, ApoA-1M production was induced with addition of IPTG (1 mM final concentration) and a temperature shift to about 37° C. or higher. Harvest was around 7 h post-induction. No kanamycin was used in the seed fermenter or the production fermenter.

### Downstream Purification

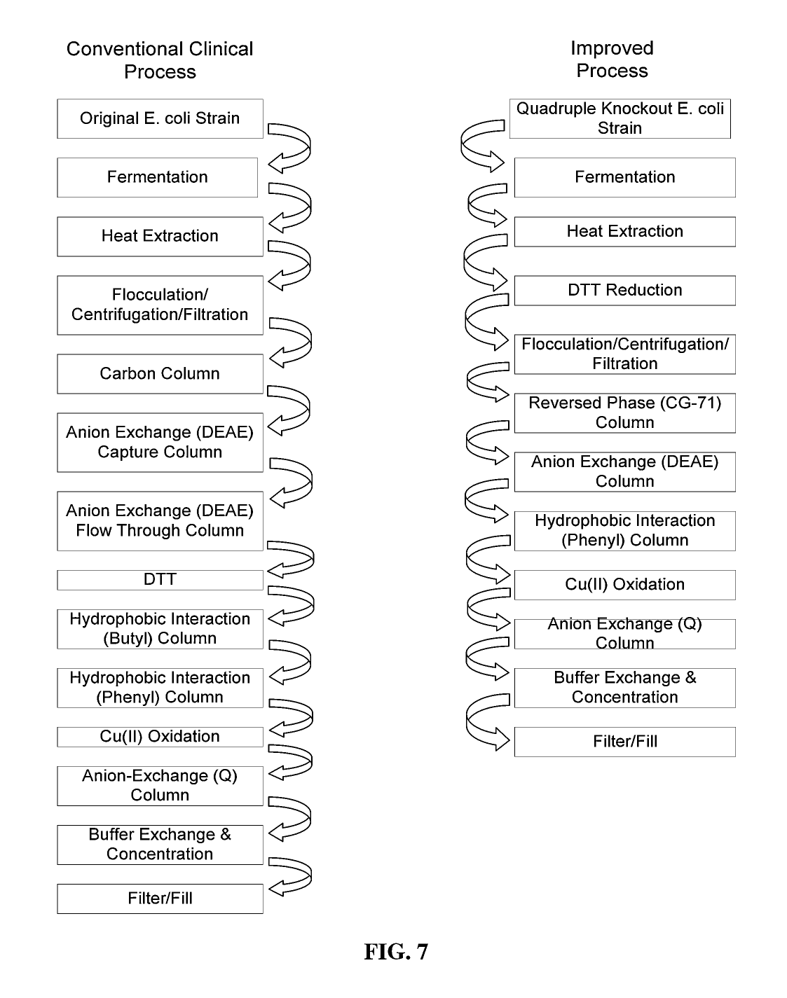

The downstream purification scheme is shown in FIG. 7, right side. At the end of the fermentation step, heat extraction is used to extract ApoA-1M from the cells, followed by flocculation, centrifugation and filtration to remove solids. The main modifications include a) earlier reduction of rApoA-1M for improved downstream processing by lowering rApoA-1M dimer and rApoA-1M disulfide linked heterodimers, and b) increase dilution of the cell broth with water for operational efficiency of these steps due to higher cell density fermentation.

The carbon column used in the previous process (FIG. 7, left side) is eliminated. Low molecular weight color compounds are removed with the subsequent reverse phase chromatography step in the new process.

The first chromatography step in the new process is a reversed phase with CG-71 resin for capturing the ApoA-1M and allowing impurities in the extracted broth to flow through. This column effectively replaces the DEAE capture column in the prior process. The next step is the bind/elute DEAE column, which further purifies ApoA-1M from both process-related impurities (DNA, HCPs, endotoxins) as well as some product related impurities (truncated species). This step represents an enhancement over the flow-through DEAE step in the old process, which functions primarily as a DNA/endotoxin reduction step.

The Butyl HIC column in the old process is also eliminated in the new process. The role of the Butyl column was to reduce HCPs for the final product to meet the HCP specification. In the new process, the combination of genetic modifications to remove the key HCP contaminants along with the reverse phase CG-71 and the bind/elution DEAE chromatography steps is effective to reduce the HCP level below what was accomplished with the Butyl column in the old process.

The next chromatography step in the new process is the Phenyl HIC column. Even though this step was also used in the old process, its operation has been optimized to specifically remove impurity fractions that have been associated with cytokine response. Key factors for this separation include protein loading and proper wash/elution conditions.

The oxidation step uses the same chemical ingredients as in the old process (Cu(II) at 50° C.) but it has been optimized with respect to protein concentration and Cu(II) level, resulting in shorter reaction time (8-10 min), higher dimer yield (85-90%) and lower aggregates (<3%).

The Q chromatography operation was optimized to increase protein loading from 3-4 g/l to 6-8 g/l resin with improved separation of monomer, dimer and aggregates.

### Methods of Treatment

The present disclosure further provides methods and formulations for the treatment or prevention of acute coronary syndromes, including unstable angina, ST-segment elevation myocardial infarction and non Q wave myocardial infarction. Safe and effective doses for the pharmaceutical formulations described herein have been determined for the treatment and prevention of acute coronary syndromes.

Based on current understanding, a framework has emerged which has reorganized clinical presentations, now termed acute coronary syndromes (“ACS”). Acute coronary syndromes comprises unstable angina, Q wave and non-ST-segment elevation myocardial infarction and is a major cause of morbidity and mortality, especially within the first 24 hours after presentation. (Schoenhagen et al., 2000, Circulation 101: 598-603). ACS is an ischemic discomfort that presents without ST segment elevation on an electrocardiograph. The ischemia often develops into unstable angina, Q wave and non-Q wave myocardial infarction. (Antman and Braunwald, “Acute Myocardial Infarction” in Heart Disease, A Textbook of Cardiovascular Medicine, 6.sup.th edition, vol. 2, Braunwald et al., eds, 2001, W. B. Saunders Company, Philadelphia).

The methods and formulations herein provide unique and effective approaches to the treatment of atherosclerosis and acute coronary syndromes. rApoA-1M produced as disclosed herein and rApoA-1M:lipid complexes, including rApoA-1M:phospholipid complexes, or pharmaceutical formulations thereof provide a non-surgical therapy that reverses the pathophysiologic basis of the disease. The methods include administration of rApoA-1M, rApoA-1M:lipid complexes, rApoA-1M:phospholipid complexes or pharmaceutical formulations thereof that provide HDL therapy which promotes cholesterol efflux, reverse cholesterol transport and reduces atherosclerotic plaque.

Included are methods for the treatment of acute coronary syndromes. In certain embodiments, rApoA-1M:lipid complexes, rApoA-1M:phospholipid complexes or pharmaceutical formulations thereof, as described below, can be administered in a dose of about 1 mg (protein)/kg to about 100 mg (protein)/kg, preferably a dose of 1 mg (protein)/kg to 50 mg (protein)/kg; most preferably, 15 mg/kg or 45 mg/kg.

Methods include the use of improved formulations for the treatment or prevention of acute coronary syndromes including alleviation or amelioration of the signs or symptoms of acute coronary syndromes. Embodiments include, but are not limited to the treatment or reduction of coronary atherosclerosis, the promotion of cholesterol efflux from affected vessels, the promotion of reverse cholesterol transport, decreased atheroma volume in an affected vessel such as a coronary artery, a decrease in total plaque volume of an affected vessel, a decrease in the average maximal plaque thickness in an affected vessel, a decrease in average maximal atheroma thickness, a decrease in plaque volume in least percent plaque area, a decrease in the greatest percent plaque area, and increased mean coronary luminal diameter in an affected vessel. Further embodiments include improved treatments in which a subject receiving the disclosed formulations have decreased angiographic lesions as compared with subjects not receiving the formulations, in which a subject exhibits a regression in pre-existing lesions, or in which a subject exhibits patency of an occluded vessel or maintenance of patency of an occluded vessel.

It is understood by those of skill in the art that the actual dose of the formulations described herein can vary with the height, weight, age, or severity of illness of the subject, the presence of concomitant medical conditions and the like. For example, an elderly subject with compromised renal or liver function can be treated with a dose of rApoA-I Milano:lipid complex or rApoA-1M:phospholipid complex that is at the lower range of the about 1 mg/kg dose (e.g., 0.8 mg/kg or 0.9 mg/kg). A subject with severe acute coronary syndromes that is obese with good renal and liver function can be treated with a dose of rApoA-I Milano:lipid complex or rApoA-1M:phospholipid complex that is, for example, at the upper range of the about 100 mg/kg dose (e.g., 120 mg/kg, 119 mg/kg, 118 mg/kg, 115 mg/kg and the like). The dosages of the formulations described herein have been shown to be effective to achieve the intended purpose. These doses achieve a range of circulating concentrations that include the effective dose with an acceptable risk to benefit profile.

Methods also include treating or preventing acute coronary syndromes with a dosing administration schedule sufficient to treat acute coronary syndromes in a subject in need of such treatment. In certain embodiments, the rApoA-1M:lipid complex, rApoA-1M:phospholipid complex or pharmaceutical formulation thereof, as described below, can be administered about every day, about every other day, about every 3 days, about every 4 days, about every 5 days, about every 6 days, about every 7 days, about every 8-10 days or about every 11-14 days. This time period is also referred to as the dosing interval or interval. In certain embodiments the dosing interval can be every month, every six months, every 12 months, every 18 months or every 24 months. In certain embodiments, the rApoA-1M, lipid complex or pharmaceutical formulation thereof can be administered about every 7 days.

In certain embodiments, administration of rApoA-1M:lipid complexes, rApoA-1M:phospholipid complexes or pharmaceutical formulations thereof can be a one-time administration. In certain embodiments, administration can continue for about 1 week, about 2 weeks, about 3 weeks, about 4 weeks, about 5 weeks, about 6 weeks, about 7-12 weeks, about 13-24 weeks, about 52 weeks or continued for the life of the subject. This time period is also referred to as the dosing duration, treatment duration or duration.

Thus, a dosing administration schedule can be, for example, a pharmaceutical formulation of an rApoA-1M:lipid complex or rApoA-1M:phospholipid complex administered about every 7 days for about 6 weeks. In certain embodiments, the dosing interval can continue intermittently after about 52 weeks. For example, a subject can be treated once a week for about 52 weeks and then treated about 3 to about 4 times over the following year. In certain embodiments, administration of a pharmaceutical formulation comprising rApoA-1M:lipid complex or rApoA-1M:phospholipid complex is about every 7 days for about 5 weeks. Other dosing administration schedules using various dosing intervals and durations as needed in a particular embodiment are also contemplated.

The dose of rApoA-1M:lipid complex or rApoA-1M:phospholipid complex or pharmaceutical formulations thereof can vary over the duration of treatment. For example, a subject can be treated with 45 mg/kg of a pharmaceutical formulation of an rApoA-1M:phospholipid complex once weekly for 3 weeks, and then treated with 15 mg/kg of a pharmaceutical formulation of an rApoA-1M:phospholipid complex once every four months or once per year for the lifetime of the subject. Such intermittent doses can be administered to maintain the patency of a vessel. Intermittent doses during the lifetime of the subject to maintain a reduced atheroma volume and increased vessel lumen are contemplated.

The methods and formulations of the present disclosure can be used in conjunction with surgical intervention, i.e., before, during or after surgery. Surgical intervention can include angioplasty, intravascular ultrasound, coronary artery bypass graft (CABG), coronary angiography, implantation of vascular stents, percutaneous coronary intervention (PCI) and/or stabilization of plaques. In certain embodiments, the methods provide for dosing of rApoA-1M:lipid complex, rApoA-1M:phospholipid complex or pharmaceutical formulations thereof before or after surgical intervention to open an occluded vessel, or reduce atherosclerotic plaque in a vessel. Surgical intervention refers to manual, non-pharmacologic or operative methods used for diagnosis, imaging (radiology) prevention or treatment of disease or a condition. For example, intravascular ultrasound (IVUS) and coronary angiography are procedures that can provide a quantitative assessment of plaque burden (diagnostic purpose), angiography can provide images of vessels (radiologic purpose) and angioplasty can open an occluded vessel (treatment purpose). All are included as surgical interventions as used herein.

### Methods of Administration

The rApoA-1M:lipid complexes, rApoA-1M:phospholipid complexes or pharmaceutical formulations thereof can be administered by any suitable route known to those of skill in the art that ensures bioavailability in the circulation. Any route of administration that provides a therapeutically effective amount of the formulations of the disclosure can be used. The route of administration can be indicated by the type of pharmaceutical formulation. For example, injectable formulations can be administered parenterally, including, but not limited to, intravenous (IV), intramuscular (IM), intradermal, subcutaneous (SC), intracoronary, intraarterially, pericardially, intraarticular and intraperitoneal (IP) injections. (See, e.g., Robinson et al., 1989, In: Pharmacotherapy: A Pathophysiologic Approach, Ch. 2, pp. 15-34, incorporated herein by reference in its entirety.)

In certain embodiments, the rApoA-1M:lipid complexes, rApoA-1M:phospholipid complexes or pharmaceutical formulations thereof can be administered parenterally or intravenously. An intravenous administration can be as a bolus, for example, administered over about 2-3 minutes or by continuous infusion by means of a pump over about 1 hour or continuously infused, over about 24 hours. In certain embodiments, the infusion can be over about 1 to about 3 hours.

The methods provide for intravenous infusion of the pharmaceutical formulations described herein. Any suitable vessel can be used as the infusion site, including peripheral vessels such as at the antecubital fossa of the arm or a central line into the chest. In certain embodiments, administration can be by a mechanical pump or delivery device, e.g., a pericardial delivery device (PerDUCER®) or cardiopulmonary bypass machine.

### Lipid Complexes

In certain embodiments, the methods of the disclosure comprise administration of lipid complexes of rApoA-1M, such as rApoA-1M:phospholipid complexes, and pharmaceutical formulations of the complexes. Efficacy can be enhanced by complexing lipids to rApoA-1M. Typically, the lipid is mixed with the rApoA-1M prior to administration. rApoA-1M and lipids can be mixed in an aqueous solution in appropriate ratios and can be complexed by methods known in the art including freeze-drying, detergent solubilization followed by dialysis, microfluidization, sonication, and homogenization. Complex efficiency can be optimized, for example, by varying pressure, ultrasonic frequency, or detergent concentration. An example of a detergent commonly used to prepare rApoA-1M:phospholipid complexes is sodium cholate. In some cases, however, it is preferable to administer the rApoA-1M alone, essentially lipid-free, to treat or prevent acute coronary syndromes.

The rApoA-1M:lipid complex or rApoA-1M:phospholipid complex can be in solution with an appropriate pharmaceutical diluent. In other embodiments, freeze-dried or lyophilized preparations of rApoA-1M:lipid complexes or rApoA-1M:phospholipid complexes can be hydrated or reconstituted with an appropriate pharmaceutical diluent prior to administration. In yet other embodiments, the rApoA-1M:lipid complexes or rApoA-1M:phospholipid complexes can be frozen preparations that are thawed until a homogenous solution is achieved prior to administration to a subject in need thereof.

The lipid can be any suitable lipid known to those of skill in the art. Non-phosphorus containing lipids can be used, including stearylamine, dodecylamine, acetyl palmitate, (1,3)-D-mannosyl-(1,3)diglyceride, aminophenylglycoside, 3-cholesteryl-6′-(glycosylthio)hexyl ether glycolipids, N-(2,3-di(9-(Z)-octadecenyloxy))-prop-1-yl-N,N,N-trimethylammonium chloride and fatty acid amides.

In certain embodiments, the lipid is a phospholipid. The phospholipid can be obtained from any source known to those of skill in the art. For example, the phospholipid can be obtained from commercial sources, natural sources or by synthetic or semi-synthetic means known to those of skill in the art (Mel'nichuk et al., 1987, Ukr. Biokhim. Zh. 59 (6):75-7; Mel'nichuk et al., 1987, Ukr. Biokhim. Zh. 59 (5):66-70; Ramesh et al., 1979, J. Am. Oil Chem. Soc. 56 (5):585-7; Patel and Sparrow, 1978, J. Chromatogr. 150 (2):542-7; Kaduce et al., 1983, J. Lipid Res. 24 (10):1398-403; Schlueter et al., 2003, Org. Lett. 5 (3):255-7; Tsuji et al., 2002, Nippon Yakurigaku Zasshi 120 (1):67P-69P). The phospholipid can be any phospholipid known to those of skill in the art. For example, the phospholipid can be a small alkyl chain phospholipid, phosphatidylcholine, egg phosphatidylcholine, soybean phosphatidylcholine, dipalmitoylphosphatidylcholine, soy phosphatidylglycerol, egg phosphatidylglycerol, distearoylphosphatidylglycerol, dimyristoylphosphatidylcholine, distearoylphosphatidylcholine, dilaurylphosphatidylcholine, 1-myristoyl-2-palmitoylphosphatidylcholine, 1-palmitoyl-2-myristoylphosphatidylcholine, 1-palmitoyl-2-stearoylphosphatidylcholine, 1-stearoyl-2-palmitoylphosphatidylcholine, dioleoylphosphatidylcholine, 1-palmitoyl-2-oleoylphosphatidylcholine, 1-oleoyl-2-palmitylphosphatidylcholine, dioleoylphosphatidylethanolamine, dilauroylphosphatidylglycerol, phosphatidylserine, phosphatidylethanolamine, phosphatidylinositol, phosphatidylglycerol, diphosphatidylglycerol, dimyristoylphosphatidylglycerol, dipalmitoylphosphatidylglycerol, distearoylphosphatidylglycerol, dioleoylphosphatidylglycerol, phosphatidic acid, dimyristoylphosphatidic acid, dipalmitoylphosphatidic acid, dimyristoylphosphatidylethanolamine, dipalmitoylphosphatidylethanolamine, dimyristoylphosphatidylserine, dipalmitoylphosphatidylserine, brain phosphatidylserine, sphingomyelin, sphingolipids, brain sphingomyelin, dipalmitoylsphingomyelin, distearoylsphingomyelin, galactocerebroside, gangliosides, cerebrosides, phosphatidylglycerol, phosphatidic acid, lysolecithin, lysophosphatidylethanolamine, cephalin, cardiolipin, dicetylphosphate, distearoyl-phosphatidylethanolamine and cholesterol and its derivatives.

The phospholipid can also be a derivative or analogue of any of the above phospholipids. In certain embodiments, the rApoA-1M:phospholipid complex can comprise combinations of two or more phospholipids. In certain embodiments, the phospholipid is 1-palmitoyl-2-oleoyl phosphatidylcholine (POP) or (1-palmitoyl-2-oleoyl-sn-glycero-3-phosphocholine) (POPC). The rApoA-1M:POPC complex can comprise about a one to one ratio by weight of rApoA-1M:POPC.

The complex comprising rApoA-1M and a lipid can comprise any amount of lipid, preferably phospholipid, and any amount of rApoA-1M effective to treat or prevent acute coronary syndromes. In certain embodiments, the rApoA-1M can comprise a complex of rApoA-1M and a phospholipid in a ratio of about one to about one by weight. However, the rApoA-1M can comprise complexes with other ratios of phospholipid to rApoA-1M such as about 100:1, about 10:1, about 5:1, about 3:1, about 2:1, about 1:1, about 1:2, about 1:3, about 1:5, about 1:10 and about 1:100 (wt of protein/wt of lipid). A ratio by weight of between about 1:0.5 to about 1:3 (wt of protein/wt of lipid) or a ratio of about 1:0.8 to about 1:1.2 (wt of protein/wt of lipid) is certain to produce the most homogenous population and are exemplified for purposes of producing stable and reproducible batches. In certain embodiments, a ratio of rApoA-1M to phospholipid of 1:0.95 (wt of protein/wt of lipid) can be used. Additional lipids suitable for use in the methods of the disclosure are well known to persons of skill in the art, and are cited in a variety of well known sources, e.g., McCutcheon's Detergents and Emulsifiers and McCutcheon's Functional Materials, Allured Publishing Co., Ridgewood, N.J., both of which are incorporated herein by reference.

Generally, it is desirable that the lipids are liquid-crystalline at 37° C., 35° C., or 32° C. Lipids in the liquid-crystalline state typically accept cholesterol more efficiently than lipids in the gel state. As subjects typically have a core temperature of about 37° C., lipids that are liquid-crystalline at 37° C. are generally in a liquid-crystalline state during treatment.

### Preparation of Lipid Complexes

The rApoA-1M:lipid complexes can be made by any method known to one of skill in the art. In some cases it is desirable to mix the lipid and rApoA-1M prior to administration. Lipids can be in solution or in the form of liposomes or emulsions formed using standard techniques such as homogenization, sonication or extrusion. Sonication is generally performed with a tip sonifier, such as a Branson tip sonifier, in an ice bath. Typically, the suspension is subjected to several sonication cycles. Extrusion can be carried out by biomembrane extruders, such as the Lipex Biomembrane Extruder™ (Lipex Biomembrane Extruder, Inc. Vancouver, Canada). Defined pore size in the extrusion filters can generate unilamellar liposomal vesicles of specific sizes. The liposomes can also be formed by extrusion through an asymmetric ceramic filter, such as a Ceraflow Microfilter™, commercially available from the Norton Company, Worcester Mass. or through a polycarbonate filter or other types of polymerized materials (i.e. plastics) known to those of skill in the art.

An rApoA-1M:lipid complex can be prepared in a variety of forms, including, but not limited to vesicles, liposomes or proteoliposomes. A variety of methods well known to those skilled in the art can be used to prepare the rApoA-1M:lipid complexes. A number of available techniques for preparing liposomes or proteoliposomes can be used. For example, rApoA-1M can be co-sonicated (using a bath or probe sonicator) with the appropriate lipid to form lipid complexes. In certain embodiments, rApoA-1M can be combined with preformed lipid vesicles resulting in the spontaneous formation of an apolipoprotein:lipid complex. In another embodiment, the rApoA-1M can also be made by a detergent dialysis method; e.g., a mixture of rApoA-1M, lipid and a detergent such as cholate can be dialyzed to remove the detergent and reconstituted to make the lipid complexes. (See, e.g., Jonas et al., 1986, Methods Enzymol. 128, 553-82).

In another embodiment, the lipid complexes can be made by co-lyophilization, as described in U.S. Pat. Nos. 6,287,590 and 6,455,088, the contents of which are hereby incorporated by reference in their entirety. Other methods are disclosed, for example, in U.S. Pat. Nos. 6,004,925, 6,037,323 and 6,046,166, incorporated herein by reference in their entireties. Other methods of preparing rApoA-1M:lipid complexes will be apparent to those of skill in the art.

In a certain embodiment, the lipid complexes can be made by homogenization. In certain embodiments, the making of rApoA-1M:lipid complexes begins when recombinant rApoA-1M is diluted to a concentration of 15 mg/mL in solution with water for injection. Sodium phosphate is added to a final concentration of 9-15 mM phosphate and to adjust the pH to between about 7.0 and about 7.8. Mannitol is added to achieve a concentration of about 0.8% to about 1% mannitol (w/v). Then POPC is added to achieve a mixture of about 1:0.95 (wt protein/wt lipid) of rApoA-1M dimer to POPC. The mixture is stirred at 5000 rpm for about 20 minutes using an overhead propeller and an Ultra Turrax while maintaining the temperature between 37° C. to 43° C. The feed vessel is stirred continuously at 300 rpm while the temperature is maintained between 32° C. to 43° C. with in-line heat exchangers (Avestin, Inc.). Homogenization for the first 30 minutes is carried out at 50 MPa (7250 psi) and thereafter, the pressure is maintained at 80-120 MPa (11600-17400 psi) until in-process testing by gel permeation chromatography demonstrates the % AUC of >70% between protein standards. The complexes may also be made as 10 mg/mL, 11 mg/mL, 12 mg/mL, 13 mg/mL and 14 mg/mL formulations wherein the weight is that of protein.

### Combination Therapy

The rApoA-1M or lipid complexes or pharmaceutical formulations thereof can be used alone or in combination therapy with other interventions in the methods of the present disclosure. Such therapies include, but are not limited to simultaneous or sequential administration of other drugs. The co-administration of another drug can be to treat, prevent or ameliorate accompanying diseases, conditions, disorders or symptoms, for example, antiarrhythmic drugs administered to treat a co-existing arrhythmia. In certain embodiments, the methods provide for co-administration of drugs to treat or prevent pain accompanying acute coronary syndromes.

The rApoA-1M, or lipid complex, or pharmaceutical formulation thereof, can be administered with other pharmaceutically active drugs, including, but not limited to, alpha/beta adrenergic antagonists, antiadrenergic agents, alpha-1 adrenergic antagonists, beta adrenergic antagonists, AMP kinase activators, angiotensin converting enzyme (ACE) inhibitors, angiotensin II receptor antagonists, calcium channel blockers, antiarrhythmic agents, vasodilators, nitrates, vasopressors, inotropic agents, diuretics, anticoagulation agents, antiplatelet aggregation agents, thrombolytic agents, antidiabetic agents, antioxidants, anti-inflammatory agents, bile acid sequestrants, statins, cholesterol ester transfer protein (CETP) inhibitors, cholesterol reducing agents/lipid regulators, drugs that block arachidonic acid conversion, estrogen replacement therapy, fatty acid analogues, fatty acid synthesis inhibitors, fibrates, histidine, nicotinic acid derivatives, peroxisome proliferator activator receptor agonists or antagonists, fatty acid oxidation inhibitors, thalidomide or thiazolidinediones (Drug Facts and Comparisons, updated monthly, January 2003, Wolters Kluwer Company, St. Louis, Mo.; Physicians Desk Reference (56.sup.th edition, 2002) Medical Economics).

Other drugs singly or in combination, that can add to or can synergize the beneficial properties of the rApoA-1M, lipid complexes or pharmaceutical formulations thereof include but are not limited to: Alpha/Beta Adrenergic Antagonists (“.beta.-blockers”) such as, carvediol, (Coreg®); labetalol HCl, (Normodyne®); Antiadrenergic Agents such as guanadrel, (Hylorel®); guanethidine, (Ismelin®); reserpine, clonidine, (Catapres® and Catapres-TTS®); guanfacine, (Tenex®); guanabenz, (Wytensin®); methyldopa and methyldopate, (Aldomet®); Alpha-1 Adrenergic Antagonist such as doxazosin, (Cardura®); prazosin, (Minipress®); terazosin, (Hytrin®); and phentolamine, (Regitine®); Beta Andrenergic Antagonists such as sotalol, (Betapace AF® and Betapace®); timolol, (Blocadren®); propranolol, (InderalLA® and Inderal®); betaxolol, (Kerlone®); acebutolol, (Sectral®); atenolol, (Tenormin®); metoprolol, (Lopressor® and Toprol-XL®); bisoprolol, (Zebata®); carteolol, (Cartrol®); esmolol, (Brevibloc®); naldolol, (Corgard®); penbutolol, (Levatol®); and pindolol, (Visken®); AMP kinase activators such as ESP 31015, (ETC-1001); ESP 31084, ESP 31085, ESP 15228, ESP 55016 and ESP 24232; gemcabene (PD 72953 and CI-1027); and MEDICA 16; Angiotensin Converting Enzyme (ACE) Inhibitors such as quinapril, (Accupril®); benazepril, (Lotensin®); captopril, (Capoten®)); enalapril, (Vasotec®); ramipril, (Altace®); fosinopril (Monopril®); moexipril, (Univasc®); lisinopril, (Prinivil® and Zestril®); trandolapril, (Mavik®), perindopril, (Aceon®); and Angiotension II Receptor Antagonists such as candesaartan, (Atacand®); irbesartan, (Avapro®); losartan, (Cozaar®); valsartan, (Diovan®); telmisartan, (Micardis®); eprosartan, (Tevetan®); and olmesartan, (Benicar®); Calcium Channel Blockers such as nifedipine, (Adalat®, Adalat CC®, Procardia® and Procardia XL®); verapamil, (Calan®, CalanSR®, Covera-HS®, IsoptinSR®, Verelan® and VerelanPM®); diltiazem, (Cardizem®, CardizemCD® and Tiazac®); nimodipine, (Nimotop®); amlodipine, (Norvasc®); felodipine, (Plendil®); nisoldipine, (Sular®); bepridil, (Vascor®); isradipine, (DynaCirc®); and nicardipine, (Cardene®); Antiarrhythmics such as various quinidines; procainamide, (Pronestyl® and Procan®); lidocaine, (Xylocalne®); mexilitine, (Mexitil®); tocamide, (Tonocard®); flecamide, (Tambocor®); propafenone (Rythmol®), moricizine, (Ethmozine®); ibutilide, (Covert®); disopyramide, (Norpace®); bretylium, (Bretylol®); amiodarone, (Cordarone®); adenosine, (Adenocard®); dofetilide (Tikosyn®); and digoxin, (Lanoxin®); Vasodilators such as diazoxide, (Hyperstat IV®); hydralazine, (Apresoline®); fenoldopam, (Corolpam®); minoxidil, (Loniten®); and nitroprusside, (Nipride®); Nitrates such as isosorbide dinitrate; (Isordil® and Sorbitrate®); isosorbide mononitrate, (Imdur®, Ismo® and Monoket®); Nitroglycerin paste, (Nitrol®); various nitroglycerin patches; nitroglycerin SL, (Nitrostat®), Nitrolingual spray; and nitroglycerin IV, (Tridil®); Vassopressors such as norepinephrine, (Levophed®); and phenylephrine, (Neo-Synephrine®); Inotrophic Agents such as aminone; (Inocor®); dopamine, (Intropine®); dobutamine, (Dobutrex®); epinephrine, (Adrenalin®); isoproternol, (Isuprel®), milrinone, (Primacor®); Diuretics such as spironolactone, (Aldactone®); torsemide, (Demadex®); hydroflumethiazide, (Diucardin®); chlorothiazide, (Diuril®); ethacrynic acid, (Edecrin®); hydrochlorothiazide, (hydroDIURIL® and Microzide®); amiloride, (Midamor®); chlorthalidone, (Thalitone® and Hygroton®); bumetanide, (Bumex®); furosemide, (Lasix®); indapamide, (Lozol®); metolazone, (Zaroxolyn®); triamterene, (Dyrenium®); and combinations of triamterene and hydrochlorothiazide (Dyazide® and Maxzide®); Antithrombotics/Anticoagulants/Antiplatelet such as bivalirudin, (Angiomax®); lepirudin, (Refludan®); various heparins; danaparoid, (Orgaran®); various low molecular weight heparins; dalteparin (Fragmin®); enoxaparin (Lovenox®); tinzaparin, (Innohep®); warfarin, (Coumadin®); dicumarol, (Dicoumarol®); anisindione, (Miradone®); aspirin; argatroban, (Argatroban®); abciximab, Reopro®); eptifibatide, (Integrilin®); tirofiban, (Aggrastat®); clopidogrel, (Plavix®); ticlopidine, (Ticlid®); and dipyridamole, (Persantine®); Thrombolytics such as alteplase, (Activase®); tissue plasminogen activator (TPA), (Activase®); anistreplase, APSAC, (Eminase®); reteplase, rPA, (Retavasae®); steptokinase, SK, (Streptase®); urokinase, (Abbokinase®); Antidiabetic agents such as metformin, (Glucophage®); glipizide, (Glucotrol®); chlorpropamide, (Diabinese®); acetohexamide, (Dymelor®); tolazamide, (Tolinase®); glimepride, (Amaryl®); glyburide, (DiaBeta® and Micronase®); acarbose, (Precose®); miglitol, (Glyset®); repaflinide, (Prandin®); nateglinide, (Starlix®); rosiglitazone, (Avandia®); and pioglitazone (Actos®); Antioxidants and anti-inflammatory agents; Bile Acid Sequestrants such as cholestyramine, (LoCholest®, Prevalite® and Questran®); colestipol, (Colestid®); and colesevelam, (Welchol®); Statins (drugs that inhibit HMGCoA reductase) such as rovastatin, (Crestor®); fluvastatin, (Lescol®); atorvastatin, (Lipitor®); lovastatin, (Mevacor®); pravastatin, (Pravachol®); and simvastatin, (Zocor®); CETP inhibitors; drugs that block arachidonic acid conversion: Estrogen replacement therapy; Fatty acid analogues such as PD 72953, MEDICA 16, ESP 24232, and ESP 31015; Fatty acid synthesis inhibitors; fatty acid synthesis inhibitors; fatty acid oxidation inhibitors, ranolazine, (Ranexa®); Fibrates such as clofibrate, (Atromid-S®); gemfibrozil, (Lopid®); micronized fenofibrate capsules, (Tricor®); bezafibrate and ciprofibrate; histidine; Nicotinic Acid derivatives such as niacin extended-release tablets, (Niaspan®); Peroxisome proliferator activator receptor agonists and antagonists; thalidomide, (Thalomid®) and compounds described in U.S. Pat. Nos. 6,459,003, 6,506,799 and U.S. Application Publication Nos. 20030022865, 20030018013, 20020077316, and 20030078239 the contents of which are incorporated herein by reference in their entireties.

Other drugs singly or in combination, that can add to or can synergize the beneficial properties of the rApoA-1M or lipid complexes or pharmaceutical formulations thereof include, for example, anti-proliferative drugs like paclitaxel and topotecan, (Brehm et al. 2001, Biochemical Pharmacology, 61 (1):119-127) and anti-inflammatory drugs such as steroidal and non-steroidal anti-inflammatory agents (including cyclooxygenase-2 (COX-2) inhibitors).

### Pharmaceutical Formulations

The rApoA-1M or lipid complexes thereof can be administered in the form of a pharmaceutical formulation. A pharmaceutical formulation, as described herein, includes the addition of, for example, an acceptable diluent, excipient, vehicle or carrier. As is known in the art, the addition of one or more diluents, excipients, vehicle or carriers renders a formulation suitable for administration to a subject and can bestow other favorable properties such as extended shelf life.

The pharmaceutical formulations can utilize any appropriate pharmaceutically acceptable carriers or vehicles. In certain embodiments sucrose-mannitol is used, or Normal saline is often employed as the pharmaceutical carrier or vehicle. Other suitable carriers or vehicles include glucose, trehalose, sucrose, sterile water, buffered water, 0.45% saline (half Normal saline), and 0.3% glycine, and can further include glycoproteins such as albumin for enhanced stability. These formulations can be sterilized by conventional, well known sterilization techniques. The resulting aqueous solutions can be packaged for use or filtered under aseptic conditions and lyophilized (freeze-dried). The lyophilized preparation can then be combined with a sterile aqueous solution prior to administration.

The pharmaceutical formulations can also contain pharmaceutically acceptable excipients as required to approximate physiological conditions, such as pH adjusting and buffering agents, and tonicity adjusting agents, for example, sodium acetate, sodium lactate, sodium chloride, potassium chloride, and calcium chloride. Antibacterial agents, for example, phenol, benzalkonium chloride or benzethonium chloride, can be added to maintain sterility of a product, especially pharmaceutical formulations intended for multi-dose parenteral use. Suspending, stabilizing and/or dispersing agents can also be used in the formulations of the disclosure.

The pharmaceutical formulations can comprise the apolipoprotein (rApoA-1M) in a salt form. For example, because proteins can comprise acidic and/or basic termini and/or side chains, the rApoA-1M can be in the pharmaceutical formulations as either free acids or bases, or as pharmaceutically acceptable salts. Pharmaceutically acceptable salts can include, suitable acids capable of forming salts with rApoA-1M including, for example, inorganic acids such as hydrochloric acid, hydrobromic acid, perchloric acid, nitric acid, thiocyanic acid, sulfuric acid, phosphoric acid and the like; and organic acids such as formic acid, acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid and the like. Suitable bases capable of forming salts with rApoA-1M can include, for example, inorganic bases such as sodium hydroxide, ammonium hydroxide, potassium hydroxide and the like; and organic bases such as mono-, di- and tri-alkyl amines (e.g., triethyl amine, diisopropyl amine, methyl amine, dimethyl amine and the like) and optionally substituted ethanolamines (e.g., ethanolamine, diethanolamine and the like).

The pharmaceutical formulation can be in a variety of forms suitable for any route of administration, including, but not limited to, parenteral, enteral, topical or inhalation. Parenteral administration refers to any route of administration that is not through the alimentary canal, including, but not limited to, injectable administration (i.e., intravenous, intramuscular and the like as described herein). Enteral administration refers to any route of administration using the alimentary canal, oral or rectal including, but not limited to, tablets, capsules, oral solutions, suspensions, sprays and the like, as described herein. For purposes of this application, enteral administration also refers to vaginal routes of administration. Topical administration refers to any route of administration through the skin, including, but not limited to, creams, ointments, gels and transdermal patches, as described herein (see also, Remington's Pharmaceutical Sciences, 18.sup.th Edition Gennaro et al., eds.) Mack Printing Company, Easton, Pa., 1990).

Parenteral pharmaceutical formulations of the present disclosure can be administered by injection, for example, into a vein (intravenously), an artery (intraarterially), a muscle (intramuscularly), under the skin (subcutaneously or in a depot formulation), to the pericardium, to the coronary arteries. The injectable pharmaceutical formulations can be a pharmaceutically appropriate formulation for administration directly into the heart, pericardium or coronary arteries. In certain embodiments, the pharmaceutical formulations are infused into a peripheral vessel of a subject, e.g. at the arm or antecubital fossa. Injectable pharmaceutical formulations can be sterile suspensions, solutions or emulsions in aqueous or oily vehicles. The formulations for injection can be presented in unit dosage form, e.g., in ampules or in multidose containers, and can comprise added preservatives. Certain buffers for parenteral pharmaceutical formulations are phosphate, citrate and acetate.

In another embodiment, the pharmaceutical formulation can be provided in powder form for reconstitution with a suitable vehicle, including but not limited to sterile pyrogen free water; saline or dextrose before use. To this end, the rApoA-1M can be lyophilized, or co-lyophilized with a lipid, as described above, as appropriate. In another embodiment, the pharmaceutical formulations can be supplied in unit dosage forms and reconstituted prior to use.

For prolonged delivery, the pharmaceutical formulation can be provided as a depot preparation, for administration by implantation; e.g., subcutaneous, intradermal, or intramuscular injection. Thus, for example, the pharmaceutical formulation can be formulated with suitable polymeric or hydrophobic materials (e.g., as an emulsion in an acceptable oil) or ion exchange resins, or as sparingly soluble derivatives; e.g., as a sparingly soluble salt form of the rApoA-1M or rApoA-1M:lipid complex.

In another embodiment, the pharmaceutical formulation is a transdermal delivery system manufactured as an adhesive disc or patch that slowly releases the active ingredient for percutaneous absorption. In this embodiment, permeation enhancers can be used to facilitate transdermal penetration of the rApoA-1M. In another embodiment, the transdermal pharmaceutical formulation can further contain nitroglycerin for use in patients with angina.

For administration by inhalation, the pharmaceutical formulation can be delivered by aerosol spray from pressurized packs or via nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount, for example, a metered dose inhaler. Capsules and cartridges of e.g., gelatin for use in an inhaler or insufflator can be formulated comprising a powder mix of the rApoA-1M and a suitable powder base such as lactose or starch.

The formulations can, if desired, be presented in a pack or dispenser device that can comprise one or more unit dosage forms comprising the rApoA-1M pharmaceutical formulations. The pack can for example, comprise metal or plastic foil, such as a blister pack. The pack or dispenser device can be accompanied by instructions or labeling for administration.

In certain embodiments, the pharmaceutical formulation of the rApoA-1M or rApoA-1M:lipid complex can comprise a concentration of rApoA-1M sufficient to treat a subject in need thereof. In certain embodiments, the pharmaceutical formulation of the rApoA-1M or rApoA-1M:lipid complex can comprise a concentration of rApoA-1M of about 5 mg/mL to about 50 mg/mL. In certain embodiments, the formulations can comprise rApoA-1M in a concentration of about 10 mg/mL to about 20 mg/mL. In a more certain embodiment, the formulations can comprise rApoA-1M in a concentration of about 13 mg/mL to about 16 mg/mL. The concentration of rApoA-1M can be determined by any suitable technique known to those of skill in the art. In certain embodiments, the concentration of rApoA-1M is determined by size exclusion high performance liquid chromatography (SE-HPLC).

In certain embodiments, the pharmaceutical formulation of the rApoA-1M or rApoA-1M:lipid complex can comprise a concentration of lipid sufficient to form complexes with rApoA-1M. In certain embodiments, the lipid is a phospholipid. In certain embodiments, the lipid is POPC. In certain embodiments, pharmaceutical formulation of the rApoA-1M or rApoA-1M:lipid complex can comprise a concentration of POPC of about 1 mg/mL to about 50 mg/mL. In certain embodiments, formulations can comprise POPC in a concentration of about 5 mg/mL to about 25 mg/mL. In a more certain embodiment, the formulations can comprise POPC in a concentration of about 10 mg/mL to about 20 mg/mL, or POPC in a concentration of about 11 mg/mL to about 17 mg/mL. The concentration of POPC can be determined by any suitable technique known to those of skill in the art. In certain embodiments, the concentration of POPC is determined by high performance liquid chromatography (HPLC).

In certain embodiments, the pharmaceutical formulation of the rApoA-1M:lipid complex can comprise sucrose in an amount sufficient to make a pharmaceutically suitable formulation of rApoA-1M or rApoA-1M:lipid complex. In certain embodiments, the pharmaceutical formulations can comprise about 0.5% to about 20% sucrose, about 3% to about 12% sucrose, about 5% to about 7% sucrose, about 6.0% to about 6.4% sucrose, or 6.2% sucrose.

In certain embodiments, the pharmaceutical formulation of the rApoA-1M:lipid complex can comprise mannitol in an amount sufficient to make a pharmaceutically suitable formulation of rApoA-1M or rApoA-1M:lipid complex. In certain embodiments, the pharmaceutical formulations can comprise about 0.01% to about 5% mannitol, about 0.1% to about 3% mannitol, about 0.5% to about 2% mannitol, about 0.8% to about 1% mannitol, or 0.9% mannitol.

In certain embodiments, the pharmaceutical formulation of the rApoA-1M:lipid complex can comprise a buffer, such as a phosphate buffer for example, in an amount sufficient to make a pharmaceutically suitable formulation of rApoA-1M or rApoA-1M:lipid complex. In certain embodiments, the buffer concentration can be about 3 mM to about 25 mM, about 5 mM to about 20 mM, or about 8 mM to about 15 mM. In certain embodiments, an appropriate buffer is added to adjust the pH of the pharmaceutical formulation to a range suitable for administration to a subject. In certain embodiments, the pharmaceutical formulation can have a pH of about 6.8 to about 7.8, about 7.0 to about 7.8, about 7.2 to about 7.5, or about 7.5.

In certain embodiments, the pharmaceutical formulation of the rApoA-1M:lipid complex has an osmolality that is suitable for administration to a subject. In certain embodiments, the osmolality of the formulation can be about 200 to about 400 mOsm, about 220 to about 380 mOsm, about 260 mOsm to about 340 mOsm, about 280 mOsm to about 320 mOsm, or about 290 mOsm.

The formulations of the disclosure provide an rApoA-1M:lipid complex of sufficient purity to allow administration to a subject. In certain embodiments, the pharmaceutical formulation can comprise rApoA-1M at a purity of about 98% or more, about 96% or more, about 95% or more, about 93% or more, about 91% or more or about 90% or more. In a certain embodiment, the purity of the rApoA-1M is about 90% or more. The purity of the rApoA-1M can be determined by any suitable technique known to those of skill in the art. In certain embodiments, the purity of the rApoA-1M can be determined by size exclusion HPLC.

The formulations of the disclosure provide an rApoA-1M:lipid complex of sufficient purity of POPC to allow administration to a subject. In certain embodiments, the pharmaceutical formulation can comprise POPC at a purity of about 98% or more, about 96% or more, about 95% or more, about 93% or more, about 91% or more or about 90% or more. In a certain embodiment, the purity of the POPC is greater than about 90%. The purity of the POPC can be determined by any suitable technique known to those of skill in the art. In certain embodiments, the purity of the POPC can be determined by HPLC.

In certain embodiments, the rApoA-1M:lipid complex has lipid hydroperoxide amounts of about 10%, or less, about 8% or less, about 6% or less, about 4% or less, about 2% or less, about 1% or below detectable limits as determined by the ferrous oxidation/xylenol orange assay (Jiang, et al. 1992, Anal. Biochem 202: 384-389). In certain embodiments the Apo A-IM:POPC complex has a purity of greater than 85% (measured as % of total peak area) as determined by gel permeation chromatography. In certain embodiments, the formulation has little or no endotoxins. In certain embodiments, the formulation has endotoxins of <0.04 EU/mg rApoA-1M.

In certain embodiments, the formulation can contain an amount of particulates greater than 10 μm in size is <about 6,000 per 50 mL, vial as determined by light obscuration. In certain embodiments, the amount of particulates greater than 25 μm in size is <about 600 per 50 mL vial as determined by light obscuration.

In a certain embodiment, the rApoA-1M:lipid complex pharmaceutical formulation is made by diluting a rApoA-1M to a concentration of 15 mg/mL in solution with water for injection. Sodium phosphate is added to a final concentration of 9-15 mM phosphate and the pH is between about 7.0 and about 7.8. Mannitol is added to achieve a concentration of about 0.8% to about 1% mannitol (w/v). Then POPC is added to achieve a ratio of 1:0.95 (wt protein/wt lipid) of rApoA-1M dimer to POPC. The mixture is stirred at 5000 rpm for about 20 minutes using an overhead propeller and an Ultra Turrax, while maintaining the temperature between 37° C. to 43° C. The feed vessel is stirred continuously at 300 rpm while the temperature is maintained between 32° C. to 43° C. with in-line heat exchangers (Avestin, Inc.). Homogenization for the first 30 minutes is carried out at 50 MPa (7,250 psi) and thereafter, the pressure is maintained at 80-120 MPa (11,600-17,400 psi) until in-process testing by gel permeation chromatography demonstrates the percentage AUC of greater than about 70% between protein standards, ferritin and albumin. The osmolality of the complex is then adjusted to about 300 by the addition of 6.0% to 6.4% sucrose. The pharmaceutical formulation of the rApoA-1M:POPC complex is then sterilized by filtration through 0.22μ filters.

In another embodiment, the pharmaceutical formulation comprises about 12 to about 18 mg/mL rApoA-1M, about 11 to about 17 mg/mL 1-palmitoyl-2-oleoyl-sn-glycero-3-phosphocholine at pH 7.4, with 6.2% sucrose and 0.9% mannitol with an osmolality of about 280 mOsm to about 320 mOsm. The pharmaceutical formulation can have rApoA-1M at about 90% purity, POPC at about 97% purity. In a certain embodiment, no single impurity can be greater than about 2%.

The pharmaceutical formulations can be stored frozen (about −15° C. to about −25° C.). In certain embodiments, the formulations can be cold solutions, frozen solutions or lyophilized solutions. The formulations may be thawed and warmed to room temperature prior to administration to a subject. Gentle thawing and warming are recommended to avoid denaturation of the protein.

In an alternate embodiment, the formulations can be in sterile glass vials of about 2 mL to about 250 mL, preferably about 10 mL to about 100 mL, most preferably about 50 mL containing a pharmaceutical formulation comprising a rApoA-1M:phospholipid complex. In certain embodiments the pharmaceutical formulations can comprise about 10 mg/mL to about 15 mg/mL of the rApoA-1M:phospholipid complex in a final fill volume of about 39 to 41 mL per vial. The amount of rApoA-1M:phospholipid complex can be about 500 mg to 750 mg per 50 mL vial.

The pharmaceutical formulations can be for a single, one-time use, or can contain antimicrobial excipients, as described above, rendering the pharmaceutical formulations suitable for multiple uses, in for example a multi-use vial. In certain embodiments, the pharmaceutical formulations can be in unit-of-use packages. As is known to those of skill in the art, a unit-of-use package is a convenient, prescription size, patient ready unit labeled for direct distribution by health care providers. A unit-of-use package contains a pharmaceutical formulation in an amount necessary for a typical treatment interval and duration for a given indication. The methods and formulations herein provide for a unit-of-use package of a pharmaceutical formulation comprising, for example, an rApoA-1M:phospholipid complex in an amount sufficient to treat an average sized adult male or female with 15 mg/kg, once weekly for 5 weeks. In one embodiment, the unit-of-use package can comprise a pharmaceutical formulation comprising a rApoA-1M:phospholipid complex in an amount sufficient to treat an average sized adult subject with 45 mg/kg once weekly for 6 weeks. It will be apparent to those of skill in the art that the doses described herein are based on the subject's weight.

The pharmaceutical formulations can be labeled and have accompanying labeling to identify the formulation contained therein and other information useful to health care providers and subjects in the treatment and prevention of cardiovascular and vascular disorders, acute coronary syndromes, ischemic disorders and for the stabilization of plaques, including, but not limited to, instructions for use, dose, dosing interval, duration, indication, contraindications, warnings, precautions, handling and storage instructions and the like.

In further embodiments, the present disclosure provides kits for treating or preventing cardiovascular and vascular disorders, acute coronary syndromes, ischemic disorders and for the stabilization of plaques. The kits comprise one or more effective doses of rApoA-1M or rApoA-1M:lipid complex or pharmaceutical formulations thereof along with a label or labeling with instructions on using the rApoA-1M or rApoA-1M:lipid complex or pharmaceutical formulations thereof to treat or prevent acute coronary syndromes according to the methods of the disclosure. In certain embodiments, the kits can comprise components useful for carrying out the methods such as devices for delivering the rApoA-1M or rApoA-1M:lipid complex or pharmaceutical formulations thereof and components for the safe disposal of these devices, e.g. a sharps container. In certain embodiments, the kits can comprise rApoA-1M or rApoA-1M:lipid complex, or pharmaceutical formulations thereof, in a pre-filled syringes, unit-dose, or unit-of-use packages.

### Example 1

### Deletion of oppA and dppA

### Strains

The strain BC50 is a K12 derivative with two chromosomal markers suitable for identification (xyl, ara). BC50 is also resistant to T4 phage. Strain BC50 containing plasmid pKP1350 is used to produce ApoA-IM for manufacture of drug substance. Strain BC50* was obtained by curing BC50 (pKP1350) by growth at 42° C. in LB media without kanamycin supplementation.

### Vectors

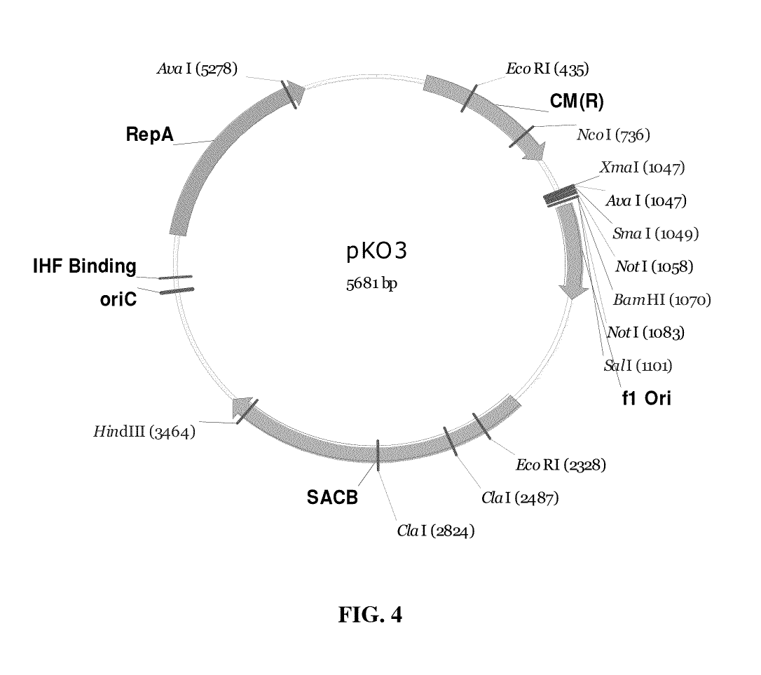

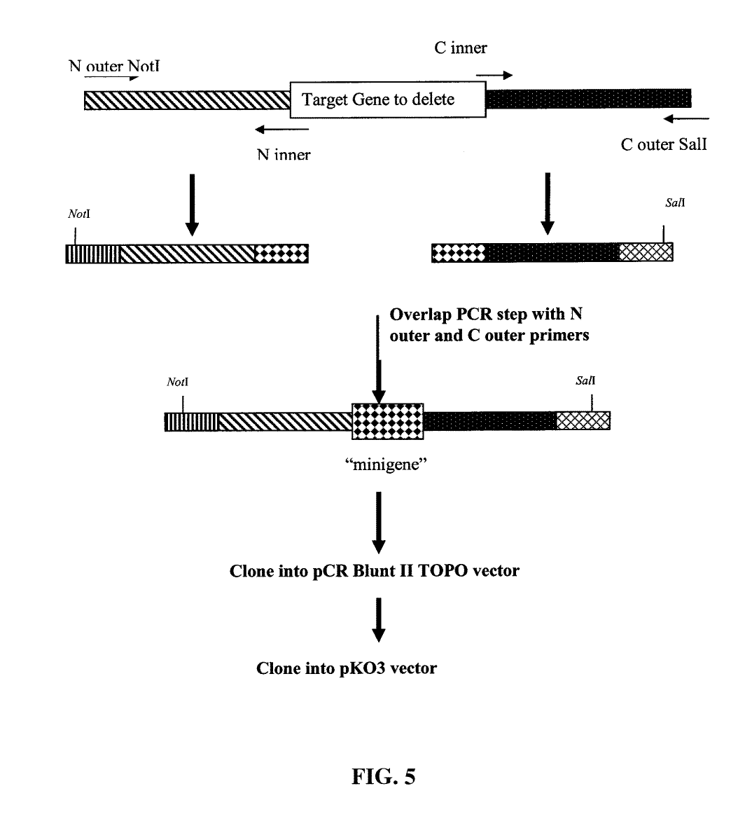

Vector plasmid pKO3 is shown in FIG. 4 (Link et al., 1997). Its origin of replication is derived from pSC101 and has a permissive temperature at 30° C. but is inactive at 42° C. The cat gene encoding chloramphenicol resistance, is used as a marker to select for chromosomal integrates and as a marker for cells harboring vector sequences after plasmid excision. The B. subtilis sacB gene that encodes levansucrase, is used to counter-select vector sequences by growing cells harboring the plasmid on medium supplemented with 5% sucrose. When expressed in E. coli on sucrose-containing media, the sacB gene is lethal (Gay et al., 1985). The Life Technologies pCR Blunt II TOPO cloning vector was used to clone the “crossover” PCR fragment (FIG. 5).

### Media

All strains were grown in LB medium (1% Tryptone, 0.5% yeast extract, 1.0% NaCl) with appropriate selection. Chloramphenicol was used at a concentration of 25 μg/mL when required. For selection against sacB, LB was supplemented with sucrose to a final sucrose concentration of 5% (w/v).

### Oligonucleotide Primers

Oligonucleotides were purchased from Sigma Genosys (The Woodlands, Tex.). The locations of PCR primers used to make the deletions are shown schematically in FIG. 5. Primers were designed using a Harvard web site program called Primer Finder at the following url: (http://arep.med.harvard.edu/primerfinder/primerfinderoverview.html)

### PCR Conditions

PCR reactions for amplification of the gene flanking regions contained 4 μM of primers, 200 μM dNTP mixture, 1 μl of genomic DNA template, 10× Expand Long buffer #1 (Roche) and 1 U of Expand Long polymerase in a 50 μl volume. The thermal cycle program was 94° C. 1 min, 88° C. 4 min, 30 cycles of 94° C. 10 sec, 65° C. 3 min, followed by 72° C. 10 min.

For generation of the overlapping deletion fragment, PCR reactions were set up as follows: 5 “N” flanking reaction, 5 “C” flanking reaction, 5 μl 10× Expand Long Buffer #1, 200 μM dNTP mixture, 0.4 μM N outer NotI.seq oligo, 0.4 μM C outer SalI.rev oligo, 1 U Expand Long polymerase in a 50 μl volume. The thermal cycle program was 94° C. 2 min, 10 cycles of 94° C. 30 sec, 60° C. 30 sec, 68° C. 45 sec, 20 cycles of 94° C. 30 sec, 65° C. 30 sec, 68° C. 45 sec with a 10 sec extension per cycle. This was followed by 7 min at 68° C.

### DNA Purification

Plasmids were purified from cultures grown overnight using Qiagen columns according to the manufacturer's recommended conditions. Qiagen kit #27106 was routinely used for purifying plasmid DNA from 1 mL volumes of cells containing TOPO plasmids. Qiagen kit #12125 was used for purifying plasmid DNA from 20 mL volumes of cells containing pKO3-derived plasmids.

Chromosomal DNA was isolated from 1 mL of cultures grown overnight using Qiagen's Dneasy kits (Catalog #69504).

Crossover PCR Deletions and Subcloning into Plasmid pKO3

The plasmid pKO3 is shown in FIG. 4. The restriction enzyme sites chosen for cloning of the “minigene” insert into pKO3 were NotI and SalI. FIG. 5 shows the strategy for the 2 steps involved in the crossover PCR deletions. In the first step, PCR amplifies the N and C terminal homologous flanking regions. Table 3 contains the sequence of the oligonucleotides used.

A PCR fragment containing approximately 500 bp upstream of the target gene and a PCR fragment containing approximately 500 bp downstream of the target gene were generated using E. coli K12 chromosomal DNA as template (FIG. 5). Primers N outer NotI and N inner were used to generate the upstream amplicon. The primers C inner and C outer SalI were used to generate the downstream amplicon.

In addition to the NotI site, the N outer primer contains the ATG start codon of the target gene followed by the minigene sequence: 5′-GTT ATA AAT TTG GAG TGT GAA GGT TAT TGC GTG; SEQ ID NO:2. The C outer primer contains the SalI site, the stop codon of the target gene and the sequence compliment of the minigene as follows: 5′-CAC GCA ATA ACC TTC ACA CTC CAA ATT TAT AAC; SEQ ID NO:3.

In the second step, the upstream and downstream fragments were annealed at their overlapping region and amplified by PCR as a single fragment, using the outer primers N outer NotI and C outer SalI. The overlapping PCR fragment was cloned into pCR Blunt II TOPO vector and sequence verified.

Once sequence verification was complete, this plasmid was digested with SalI and NotI and ligated to pKO3 digested with SalI and NotI.

TABLE 3
PCR Primers for mini-gene knock out constructs.

| Oligonucleotide | |
|---|---|
| Name | Oligonucleotide sequence 5′-3′ |
| DppA_N outer | GCGGCCGCTATCCATTAACGGATTTGTGACAG; |
| NotI.seq | SEQ ID NO: 4 |
| DppA_C outer | GCTGATGTCGACGCCACATCGGTTTATCTAA; |
| SalI.rev | SEQ ID NO: 5 |
| DppA N | CACGCAATAACCTTCACACTCCAAATTTATAACACGCATTATTCTGCTCC; |
| inner.rev | SEQ ID NO: 6 |
| DppA C | GTTATAAATTTGGAGTGTAAGGTTATTGCGTGTAATTAAAAGCCATACAAGACTG; |
| inner.seq | SEQ ID NO: 7 |
| Oppa N | GATGCAGCGGCCGCGAGGAAAGTGCTAAATAATAATCA; |
| outerNotI.seq | SEQ ID NO: 8 |
| Oppa C | GTAGTAGTCGACATTGCCAGCCCCATC; |
| outerSalI.rev | SEQ ID NO: 9 |
| Oppa N inner.rev | CACGCAATAACCTTCACACTCCAAATTTATAACCATTGTTTTTTGGACTCC; |
| | SEQ ID NO: 10 |
| Oppa C inner.seq | GTTATAAATTTGGAGTGTGAAGGTTATTGCGTGTAATGGCAATACGTGGG; |
| | SEQ ID NO: 11 |
| opp360.seq | GTATAGCTGGCAACGTTCTGTTG; |
| | SEQ ID NO: 12 |
| opp880.rev | GATCAACGTGAACTTCGTCC; |
| | SEQ ID NO: 13 |
| pKO3-L | AGGGCAGGGTCGTTAAATAGC; |
| | SEQ ID NO: 14 |
| pKO3-R | TTAATGCGCCGCTACAGGGCG; |
| | SEQ ID NO: 15 |

### DNA Sequencing

Primers M13 forward and M13 reverse were used to verify sequence of fragments cloned into the pCR Blunt II TOPO vector. The primers pKO3-L and pKO3-R were used to confirm the inserts in vector pKO3.

### Gene Deletion Procedure

The gene deletion procedure was based on that described by Link et al. (1997). The pKO3 vector containing the gene replacement “minigene” insert was transformed into the BC50* strain of E. coli. Transformants were plated on LB plates containing chloramphenicol (25 μg/ml) at 30° C. overnight. From these transformants, four colonies were picked and each inoculated into 10 mL volumes of LB containing chloramphenicol (25 μg/mL) pre-warmed to 42° C. These were incubated overnight at 42° C. with aeration. After overnight incubation, cells from each culture were streaked on pre-warmed LB chloramphenicol plates (25 μg/mL) and incubated overnight at 42° C. A colony from each streaked culture was inoculated into a 10 mL aliquot of pre-warmed LB without supplementation. Each of the 4 cultures was incubated overnight at 42° C. with aeration. Each culture was streaked onto LB containing 5% (w/v) sucrose and incubated overnight at 30° C. From the first streak, a second streak was made onto LB containing 5% (w/v) sucrose and plates incubated at 30° C. overnight. Single colonies were picked from the second set of LB sucrose plates and patched onto both LB and LB chloramphenicol plates. Between 25 and 50 colonies were routinely picked for analysis. Plates were incubated overnight at 30° C. Chloramphenicol sensitive clones were chosen as potential deletion clones. These were screened by PCR analysis for the absence of the targeted gene.

### Screening for Gene Deletions

PCR was used to screen for deletion of dppA and oppA. After sucrose counter-selection, an inoculating loop was used to transfer chloramphenicol-sensitive colonies to tubes containing 45 μl aliquots of PCR Supermix (Life Technologies catalog. No. 10572-014). Appropriate primers were added to a final concentration of 4 μM. The thermal cycle program was 94° C. 2 min, 30 cycles of 94° C. 30 sec, 72° 45 sec, 68° 30 sec, followed by 72° C. for 5 min. PCR products were analyzed on 1.2% agarose E-gels (Life Technologies).

Clones that appeared to contain the required deletion were confirmed by a second PCR analysis using purified genomic DNA as template instead of colony-derived material.

### Discussion

PCR analysis of a clone with an oppA deletion was done for confirmation of the deletion. Analysis of the BC50* parental strain and the BC50*ΔdppA strain showed that the full length oppA gene was present in both of these strains, as demonstrated by the ˜3 kb fragment obtained using the oppA gene flanking primers N outer NotI and C outer SalI. This size fragment was absent from the BC50*ΔoppA strain and from the BC50*ΔdppAΔoppA strain but was replaced by the smaller fragment of approx. 1 kb, due to absence of the oppA gene in the genome of these strains. The internal oligos opp360.seq and opp880.rev which were designed to amplify DNA from within the oppA gene showed an appropriate sized fragment of approx. 400 bp when tested with genomic DNA from the BC50* parental strain and the BC50*ΔdppA strain. This fragment was absent from the strains where the oppA gene has been deleted.

A similar PCR analysis was performed for a clone with a dppA deletion. Analysis of the BC50* parental genome showed the presence of an appropriate sized fragment amplified by dppA gene flanking oligos. This fragment was reduced in size in the strains where dppA was deleted. Further evidence that the dppA gene was deleted was demonstrated when oligos specific to a location within the dppA gene did not amplify a fragment when tested with the BC50*ΔdppA strain and the BC50*ΔdppAΔoppA strain. However, a fragment of expected size was amplified in the BC50* parental strain sample.

### Example 2

### Deletion of malE

### Strains and Vectors

The E. coli strain MC1061 (F— araD139 Δ(ara leu) 7696 ΔlacY74 galU galK hsdR hsdM+strA) was used for propagation of plasmid pKO3 (Link et al., 1997) for purification of vector. Its origin of replication is derived from pSC101 and has a permissive temperature at 30° C., but is inactive at 42° C. Plasmid pKO3 was derived from Hamilton et al. (1989). The cat gene encoding chloramphenicol resistance, is used as a marker to select for chromosomal integrates and as a marker for cells harboring vector sequences after plasmid excision. The B. subtilis sacB gene that encodes levansucrase, is used to counter-select vector sequences by growing cells harboring the plasmid on medium supplemented with 5% sucrose. When expressed in E. coli on sucrose-containing media, the sacB gene is lethal (Gay et al., 1985). The E. coli strain DH5α was used as host strain for transformations of ligation mixtures and as host for propagation of plasmid DNA for sequencing in the current example. The Life Technologies pCR Blunt II TOPO cloning vector was used to clone the “crossover” PCR fragment (FIG. 5).

### Media

All strains were grown in LB medium (1% Tryptone, 0.5% yeast extract, 1.0% NaCl) with appropriate selection. Chloramphenicol was used at a concentration of 25 μg/mL when required.

### Oligonucleotide Primers

Oligonucleotides were purchased from Sigma Genosys (The Woodlands, Tex.). The locations of PCR primers used to make the deletions are shown schematically in FIG. 5. Primers were designed using a Harvard web site program called Primer Finder: http://arep.med.harvard.edu/primerfinder/primerfinderoverview.html.

### PCR Conditions

The PCR reaction for amplification of both gene flanking regions contained 4 μM of primers, 1 μL of genomic DNA template, 10× Accuprime Pfx reaction mixture (Life Technologies) and 1 U of Accuprime Pfx polymerase in a 50 μL volume.

For amplification of the “N-terminal” flanking region the thermal cycle program was: 94° C. 2 min followed by 5 cycles of 94° C. 30 sec, 65° C. 30 sec, 68° C. 45 sec; 5 cycles of 94° C. 30 sec, 70° C. 30 sec, 68° C. 45 sec and 15 cycles of 94° C. 30 sec, 73° C. 30 sec, 68° C. 45 sec plus 5 sec extension per cycle. This was followed by 68° C. 7 min.

For amplification of the “C-terminal” flanking region, the thermal cycle program was: 94° C. 1 min, 88° C. 4 min followed by 30 cycles of 94° C. 10 sec, 65° C. 3 min. After 30 cycles the reaction was incubated at 72° C. for 10 min.

For generation of the overlapping deletion fragment, PCR reactions were set up as follows: 2 μL “N” flanking reaction, 2 μL “C” flanking reaction, 5 μL 10× Accuprime PCR mixture, 0.4 μM malE N outer NotI.seq oligo, 0.4 μM malE C outer SalI.rev oligo and 1 U Accuprime Pfx polymerase in a 50 μL volume. The thermal cycle program was 94° C. 1 min, 88° C. 4 min followed by 30 cycles of 94° C. 10 sec, 65° C. 3 min. This was followed by 10 min at 72° C.

### DNA Purification

Plasmids were purified from cultures grown overnight using Qiagen columns according to the manufacturer's recommended conditions. Qiagen kit #27106 was routinely used for purifying plasmid DNA from 1 mL volumes of cells containing TOPO plasmids. Qiagen kit #12125 was used for purifying plasmid DNA from 20 ml volumes of cells containing pKO3-derived plasmids. In order to recover the pKO3 plasmid, strains harboring pKO3 must be grown at 30° C. under chloramphenicol selection.

Crossover PCR Deletions and Sub-Cloning into Plasmid pKO3

The restriction enzyme sites chosen for cloning of the “minigene” insert into pKO3 were NotI and SalI since these enzymes do not cut in the chromosomal flanking regions of the malE gene. FIG. 5 shows the strategy for the 2 steps involved in the crossover PCR deletions. In the first step, PCR amplifies the N and C terminal homologous flanking regions. Table 4 contains the sequence of the oligonucleotides used.

A PCR fragment containing approximately 500 bp upstream of the malE gene and a PCR fragment containing approximately 500 bp downstream of the malE gene were generated using E. coli K12 chromosomal DNA as template (FIG. 5). Primers malE N outer NotI and malE N inner were used to generate the upstream amplicon. The primers malE C inner and malE C outer SalI were used to generate the downstream amplicon.

In addition to the NotI site, the N outer primer contains the ATG start codon of the malE gene followed by the minigene sequence: 5′-GTT ATA AAT TTG GAG TGT GAA GGT TAT TGC GTG; SEQ ID NO:16. The C outer primer contains the SalI site, the stop codon of the malE gene and the sequence compliment of the minigene as follows: 5′-CAC GCA ATA ACC TTC ACA CTC CAA ATT TAT AAC; SEQ ID NO:17.

In the second step, the upstream and downstream fragments were annealed at their overlapping region and amplified by PCR as a single fragment, using the outer primers N outer NotI and C outer SalI. The overlapping PCR fragment was cloned into pCR Blunt II TOPO vector and sequence verified.

Once sequence verification was complete, this plasmid was digested with SalI and NotI and ligated to pKO3 digested with SalI and NotI.

### DNA Sequencing

Primers M13 forward and M13 reverse were used to verify sequence of fragments cloned into the pCR Blunt II TOPO vector. The primers pKO3-L and pKO3-R were used to confirm the inserts in vector pKO3.

TABLE 4

| Oligonucleotide name | Oligonucleotide sequence (5′-3′) |
|---|---|
| malENinner.rev | CACGCAATAACCTTCACACTCCAAATTTATAACCATAATCTAT |
| | GGTCCTTGTTGGTGAA; |
| | SEQ ID NO: 18 |
| malENouterNotI.seq | AAGGAAAAAAGCGGCCGCTAAAGTCGATTTACCGCAGCCAGA; |
| | SEQ ID NO: 19 |
| malECinner.seq | GTTATAAATTTGGAGTGTGAAGGTTATTGCGTGTAATGCTGTG |
| | AAATGCCGGATGC; |
| | SEQ ID NO: 20 |
| malECouterSalI.rev | CGCACGCATGTCGACTGCCAGGAGCGATCTAACAACA; |
| | SEQ ID NO: 21 |
| pKO3-L | AGGGCAGGGTCGTTAAATAGC; |
| | SEQ ID NO: 22 |
| pKO3-R | TTAATGCGCCGCTACAGGGCG; |
| | SEQ ID NO: 23 |
| M13 forward | GTAAAACGACGGCCAG; |
| | SEQ ID NO: 24 |
| M13 reverse | CAGGAAACAGCTATGAC; |
| | SEQ ID NO: 25 |

### Example 3

### Deletion of ompT

### Strains

E. coli expression strain BC50* is described above. This strain was modified to have deletions for the host cell proteins dppA and oppA. The modified strain was designated BC50*ΔdppA/ΔoppA.

### Media

Bacterial stocks were cultured and maintained on standard LB broth or agar, obtained from Teknova (Hollister, Calif.). Media for culture was supplemented with antibiotic or sucrose as called for in the selection procedure. Cultures were maintained at 37° C., but shifted to 30° C. or 42° C. as called for in the selection procedure.

MIM media, [tryptone: 32 g; yeast extract: 20 g; 25×M9 salts: 20 mls; 40% glucose: 25 mLs (per liter)] was used for the expression of ApoA1M peptide.

### Primer Location and Selection

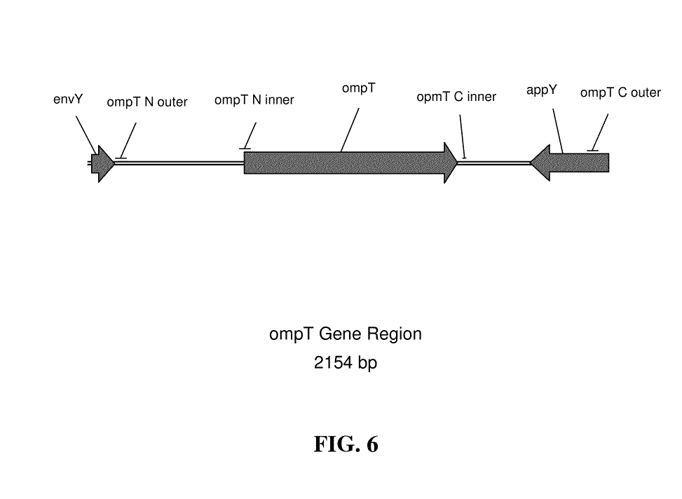

PCR primer pairs were selected by the method of Link, 1997, incorporated herein in its entirety by reference. A pair of outer primers was chosen corresponding to E. coli genomic regions flanking the ompT gene, and approximately the same distance (approx. 600 b.p.) from the N- and C-termini of the gene. The relative position of the oligomers with regard to the ompT gene is shown in FIG. 6. Sequences with relatively similar melting temperature (approx. 65-70° C.) were selected. To facilitate cloning, the outer oligos were synthesized to contain 5′ restriction sites for NotI (on the N-terminal oligo) and SalI (on the C-terminal oligo). Outer oligos were paired with a corresponding set of inner oligos, corresponding to genomic segments within (in the case of the N inner oligo) or near (in the case of the C inner, about 50 bases)) the N- and C-termini of the ompT gene. These oligos were selected to be similar in melting temperature to their outer counterparts, but lacked restriction sites. Rather, these oligos were tagged with a 33 base pair segment that served as a site of overlap-extension during the secondary PCR reaction. The outer and inner oligos are listed in Table 5.

TABLE 5
Primers Used To Generate ompT Knockout Construct

| Oligonucleotide | |
|---|---|
| name | Oligonucleotide sequence (5′-3′) |
| ompT N Outer | GCGGCCGCcaaacagcgacaaaaagtgatg; |
| | SEQ ID NO: 26 |
| ompT N Inner | CACGCAATAACCTTCACACTCCAAATTTATAACcccgcataaaagttctccattcaatc; |
| | SEQ ID NO: 27 |
| ompT C Inner | GTTATAAATTTGGAGTGTGAAGGTTATTGCGTGggaataactagccatttcaatg; |
| | SEQ ID NO: 28 |
| ompT C Outer | GTCGACtgagcggcaatggcat; |
| | SEQ ID NO: 29 |

Note:
Authentic E. coli sequence is shown in lower case, whereas restriction sites (Outer primers) and
central overlap (Inner primers) are shown in UPPER CASE.

Pcr Generation of ompT Flanking Sequences

DNA sequences corresponding to the N-terminal and C-terminal flanking regions of the ompT gene were generated using PCR from E. coli K12-derived genomic DNA, using the primers listed in Table 5. Template genomic DNA was obtained from the standard E. coli strain K12D using the DNeasy Tissue Kit (Qiagen USA, Valencia Calif.). PCR reactions were conducted using Pfx AccuPrime kit and materials (Life Technologies, Carlsbad, Calif.). Typically, 50 μL reactions were conducted using the conditions following: K12D Template DNA, approx 100 ng; Primers 400 nM each (final concentration); AccuPrime Buffer (with dNTPs) lx; Pfx DNA polymerase 1.0 unit; RNase/DNase free water (Life Technologies) to 50 μL total. Primary PCR reactions were used to generate N-terminal and C-terminal segments separately, using N Outer with N Inner primers in one reaction and C Outer with C Inner primers in a second reaction. Primary reactions were cycled as listed in Table 6. The products of primary amplification were used as templates to conduct a secondary PCR reaction that joined the N-terminal and C-terminal constructs by overlap extension, replacing the coding sequence with a short non-coding primer sequence. Reactions were typically performed by combining 5 μl of each of the N-terminal and C-Terminal primary PCR reaction product with N Outer and C Outer primers (400 nM each, cf. Table 5) in a reaction containing 1× AccuPrime Buffer and 1.0 units Pfx polymerase (both listed above). Cycle conditions were as listed in Table 6, with the exception that 60° C. (not 50° C.) was used as the annealing temperature during the initial replication stage.

TABLE 6
PCR Cycle Conditions

| Action | Temperature | Duration | Cycles |
|---|---|---|---|
| Initial Denaturation | 95° C. | 2 min | One |
| Initial Replication | 95° C. | 30 Sec | 10 Cycles |
| | 50° C. (60° C.) | 30 sec | |
| | 68° C. | 4 min | |
| Amplification | 95° C. | 30 Sec | 20 Cycles |
| | 60° C. | 30 sec | |
| | 68° C. | 4 min + 5 sec/cycle | |
| Polishing | 68° C. | 15 min | Once |
| Hold | 4° C. | Hold | Hold |

### Construct Propagation and Evaluation

Knockout constructs that had been generated by overlap extension PCR were captured by ligation into the pcDNA TOPO-Blunt plasmid (Life Technologies), using the instructions included in the kit. This plasmid construct was used for initial sequence confirmation and for propagating the construct prior to cloning into the pKO3 vector. Selected plasmid DNA containing the knockout construct was digested with the restriction endonucleases NotI and SalI (both, Roche, Indianapolis Ind.), sites which were included in the design of the flanking construct. The released knockout construct was isolated by electrophoresis on a 1% agarose gel (BioRad), excised with a fresh scalpel, purified by the Qiaex gel purification kit (Qiagen), and ligated into digested pKO3 plasmid using the Rapid Ligation Kit (Roche).

Ligated pKO3-ompT knockout plasmid was used to transform competent DH5α E. coli cells using heat shock, and cultured at 30° C. on selective media (LB with chloramphenicol at 25 μg/mL), Teknova). Individual colonies were picked for mini-prep (Qiagen) and analyzed by restriction digest with NotI and SalI. A single colony was selected that was shown by restriction digest and sequencing to contain the relevant insert. This plasmid was used to transform the BC50* E. coli expression strain, along with the BC50* Δdppa/ΔoppA strain.

### Gene Deletion Method

Transformed cell strains were selected for gene deletion using the method of Link et al, 1997, as modified by Caparon, 2010. Putative knockouts were analyzed by PCR by extracting DNA from a small inoculum and performing PCR using oligos representing the flanking regions of the gene (Table 6). As further confirmation, putative knockouts were grown overnight in LB and used to generate genomic DNA (DNeasy Kit, Qiagen) which was analyzed by secondary PCR reactions utilizing the ompT flanking primer set (as above) and the ompT internal oligomer set (Table 7).

TABLE 7
ompT Internal Primers

| Designation | Sequence (5′-3′) |
|---|---|
| ompT N Inner Fwd | cccgcataaaagttctccattcaatc; |
| | SEQ ID NO: 30 |
| ompT C Inner Rev | cattgaaatggctagttattcc; |
| | SEQ ID NO: 31 |

### Protein Expression

Protein expression in the modified cell strain was evaluated by SDS-PAGE and Western Blot. In brief, deletion constructs were transformed with the pKP1350 plasmid for ApoA1M expression, picked from LB+Kan³⁰ plates (LB agar with kanamycin at 30 μg/mL, Teknova) and grown overnight in 2 mLs of selective broth (LB+Kan³⁰). The next morning, 500 μL of the overnight culture was used to inoculate 10 mL of unsupplemented MIM broth and grown at 30° C. Duplicate inoculations were made for each strain used in the experiment. Each expression culture was monitored for growth by measuring the OD at 600 nm hourly. When a strain was shown to have reached or exceeded an OD 600 nm of 0.7, expression of ApoA1M was induced by the addition of IPTG to a final concentration of 1 mM, after which the culture temperature was shifted up to 37° C. 1 ml samples of each culture were taken immediately prior to induction (I₀ samples). ODs were monitored hourly for 4 hours post induction, at which time 1 mL samples of each culture were again taken (I₄ samples). I₀ and I₄ samples were pelleted by brief centrifugation, decanted of spent media, and stored at −20° C. until they were processed. BC50, BC50* and BC50*Δ dppA/ΔoppA parental strains were run in parallel as controls.

### Analysis of Expressed Protein

I₀ and I₄ pellets were processed by resuspending the pellet in 1×XL Sample Buffer (BioRad) at a volume equivalent to 1/10 the OD 600 nm of the original sample. Resuspended cell pellets were heated to 90° C. for 10 minutes, and then homogenized by passing through a Qiashredder column (Qiagen). Aliquots of the homogenized whole cell mixture were denatured by the addition of XL Denaturation Reagent (BioRad) to a 1× final concentration and reheating to 90° C. for 10 minutes.

Samples were run on 4-12% Bis-Tris gradient gels (BioRad). 15 μl aliquots of each sample were loaded on two gels and run in parallel, with Kaleidoscope Protein MW Standards (BioRad). Gels were run for approximately 1 hour at 200V (constant voltage). One gel of the set was stained for total protein content (SafeStain, Life Technologies), and the other was transferred to nitrocellulose membrane (Criterion, BioRad) for Western blot.

The blotting procedure used was essentially as described by Caparon, 2010. The membrane was blocked using 10% non-fat milk in Tris Buffered Saline (1×TBS, BioRad) containing 0.1% Tween 20 (BioRad). The blot was incubated overnight at 4° C. in primary antibody solution (1:2000 dilution of anti human ApoA [Rockland catalog number 600-101-109] in 2% non-fat milk, 1×TBS, 0.1% Tween 20). After washing, the blot was incubated with secondary antibody (1:10,000 dilution of rabbit anti goat IgG (H+L) [Rockland catalog number 605-4302], in 2% non-fat milk, 1×TBS, 0.1% Tween 20) for 1 hour at room temperature with constant shaking. The blot was again washed and then incubated with Super Signal West Pico Chemiluminescent Substrate (Pierce), in accordance to the manufacturer's instructions, and used to make film exposures of the blot's chemiluminescence. 1-5 second exposures were generally adequate to show the expression of ApoA 1M in all cell strains tested.

### OmpT Knockout Fragments

Individual PCR reactions were run to generate the N-terminal and C-terminal portions of the ompT knockout fragment. Agarose gels of 0.8% were used to visualize the PCR-generated N-terminal fragment, the C-terminal fragment, and the overlap extension PCR product generated in the secondary PCR reaction. The approximately 1 kb fragment representing the overlap-extension N-terminal/C-terminal knockout was subcloned into pcDNA-TOPO Blunt for capture and analysis. pcDNA-TOPO Blunt subclones were analyzed by mini-prep restriction digest. Of the ten clones represented in this analysis, only one showed an appropriately sized DNA insert. This colony was propagated and used for sequencing and subcloning into pKO3.

pKO3 Subclones

Insert from the pcDNA-TOPO clone of ompT flanking regions was confirmed by sequencing, and digested with NotI and SalI for gel purification. Purified insert was ligated into compatible ends in a digested pKO3 vector, forming the pKO3-ompTKO plasmid. The ligated plasmid was used to transform E. coli strain DH5α cells. Colonies were picked for mini prep and restriction analysis. Clone 1 from this group was sequenced. It was used to transform competent BC50* and BC50* ΔdppA/ΔoppA cells, which were then taken through the gene deletion procedure.

### Gene Deletion and Knockout Selection

Transformed BC50* and BC50* Δdppa/ΔdoppA cells were selected for deletional recombination using the method of Link, 1997 as modified by Caparon, 2010. The resultant colonies were selected by chloramphenicol sensitivity. Altogether, 19 (of 48 total colonies) were selected for further analysis.

PCR Analysis of Putative Knockouts in BC50*ΔdppA/ΔoppA

Colonies that had been shown to be chloramphenicol sensitive were sampled for PCR analysis. In the initial screen, the ompT “outer” primer pair was used (Table 5), expecting an 1118 bp deletion fragment to be seen instead of the 2073 bp fragment that would be present if the ompT coding sequence were intact. ompT had been demonstrably knocked out in only 4 of the 19 colonies of the BC50* ΔdppA/ΔoppA cells selected for chloramphenicol sensitivity. Isolated Genomic DNA from the positive colonies was analyzed by PCR This DNA showed a clear shift in size in the putative knockouts with the flanking primers when compared with the parental strain, and an absence of amplification product when using the internal primer pair. This pattern is entirely consistent with a deletion of the ompT gene.

### Other BC50* Knockouts

In parallel, BC50* parental cells (that is, BC50* with no other known deletions or modifications) were also deleted for ompT, by the same method that was applied to the dppA/oppA deleted BC50* cells. PCR analysis was also done with genomic DNA from putative BC50* ompT knockout strains, using the ompT “outer” primer set. A single clone in this set was propagated for further use.

ApoA1M Expression in ompT Knockout Strains

Knockout constructs were transformed with plasmid pKP1350 and cultured to express ApoA1M as means of comparing relative expression levels with those of parental strains. Relative growth of the expression cultures were measured by their optical density at 600 nm wavelength. Harvested cells from the I₀ and I₄ time points were also analyzed by SDS PAGE and Western Blot. Cumulatively, the growth curves and protein production of the deletion construct strains were comparable to their respective parental strains.

### Example 4

### Pharmaceutical Compositions

The recombinant ApoA-1M produced from the quadruple knockout cell line and purified as described herein was used to produce a pharmaceutically acceptable formulation. This formulation included 15 mg/ml rApoA-1M, 14.25 mg/ml POPC, 40 mg/ml sucrose, 20 mg/ml mannitol, 12 mM phosphate buffer at pH 7.4 and water. The formulation was prepared by reconstituting a lyophilized product.

For purposes of comparison, the prior art formulation was used in a formulation containing 14.5 mg/ml liquid rApoA-1M, 13.8 mg/ml POPC, 61.95 mg/ml sucrose, 7.94 mg/ml mannitol, 9-12 mM phosphate buffer at pH 7.4 and water

The pharmaceutical product, called ETC-000216 by the inventors is a complex of recombinant ApoA-IM (rApoA-IM) and a naturally occurring phospholipid intended for the treatment of atherosclerosis. A study was performed to assess and compare the potential toxicity, IL-6 response, and toxicokinetics (TK) after intravenous (IV) administration of ETC-000216 produced by prior art methods in prior art method and by the improved processes disclosed herein. The formulation was administered to monkeys at doses of 300 mg/kg every other day for 2 weeks. The potential for reversibility of any toxic changes was also assessed after a 4-week recovery period. This study consisted of 3 groups of 4 main study animals per sex per group and 2 recovery animals per sex per group. Group 1 received the vehicle, 0.9% Sodium Chloride Injection, USP. Groups 2 and 3 received 300-mg/kg doses of ETC-000216 produced via old or new methods, respectively, at concentrations of 14.5 or 15 mg/mL, respectively. All doses were administered at a volume of 21.5 mL/kg.

Toxicity was evaluated by monitoring clinical observations, body weights, food consumption, physical and ophthalmic examination observations, electrocardiography, body temperature, indirect blood pressure and heart rate, respiration rate, serum IL-6 concentration, and clinical pathology parameters (hematology, coagulation, serum chemistry, and urinalysis). Blood samples were collected at protocol-specified time points, processed to serum, and subjected to concentration and TK analysis. On Day 15, 4 animals per sex per group were euthanized and subjected to comprehensive necropsy and tissue collection; organ weights were also measured. On Day 42, all remaining animals (2 per sex per group) were euthanized and subjected to the same pathology procedures. Collected tissues were evaluated with light microscopy, and bone marrow smears were prepared.

The mean TK parameters are summarized below:

TABLE 8

| Dose | Study | | Cmax (μg/ml) | | | ↑max (h) | | | AUC (μg*h/ml) | | |
|---|---|---|---|---|---|---|---|---|---|---|---|
| (mg/kg) | Day | Gender | Mean | S.D. | N | Mean | S.D. | N | Mean | S.D. | N |
| 300 | 1 | Male | 7420 | 188 | 6 | 2.0 | 0.0 | 6 | 98100 | 13000 | 6 |
| (prior | | Female | 7930 | 728 | 6 | 2.0 | 0.0 | 6 | 99400 | 10700 | 6 |
| art) | | Overall | 7680 | 574 | 12 | 2.0 | 0.0 | 12 | 98700 | 11300 | 12 |
| | 13 | Male | 5750 | 968 | 6 | 2.0 | 0.0 | 6 | 58800 | 9280 | 6 |
| | | Female | 5690 | 567 | 6 | 2.0 | 0.0 | 6 | 55600 | 9380 | 6 |
| | | Overall | 5720 | 757 | 12 | 2.0 | 0.0 | 12 | 55600 | 9380 | 12 |
| 300 | 1 | Male | 9580 | 2150 | 6 | 2.0 | 0.0 | 6 | 116000 | 30800 | 6 |
| rAPO | | Female | 9040 | 2180 | 6 | 2.0 | 0.0 | 6 | 103000 | 16600 | 6 |
| A-1M | | Overall | 9310 | 2090 | 12 | 2.0 | 0.0 | 12 | 110000 | 24500 | 12 |
| | 13 | Male | 9040 | 2170 | 6 | 2.0 | 0.0 | 6 | 106000 | 37200 | 6 |
| | | Female | 7050 | 1140 | 6 | 2.0 | 0.0 | 6 | 71900 | 9060 | 6 |
| | | Overall | 8050 | 1950 | 12 | 2.0 | 0.0 | 12 | 88800 | 31200 | 12 |

Cₘₐₓ = maximum serum ETC-000216 concentration;
↑ₘₐₓ = time of maximum serum ETC-000216 concentration;
AUC₍₀₋₂₄₎ = area under the concentration versus time curve from time zero to 24 hours after dosing;
S.D. = standard deviation;
Overall = combined male plus female.

There were no apparent gender-related differences in ETC-000216 exposure. Systemic exposure (as assessed by maximum serum ETC-000216 concentration [Cₘₐₓ] and area under the concentration versus time curve from time zero to 24 hours after dosing [AUC₍₀_{_{—}}₂₄₎]) on Days 1 and 13 was higher for the 300-mg/kg dose as disclosed herein than for the 300-mg/kg prior art dose. Systemic exposure was lower on Day 13 than on Day 1. No detectable serum concentrations of ETC-000216 were observed on Day 42 in any of the recovery phase animals.

This example thus demonstrates that the novel formulations of the present disclosure provide a surprisingly higher maximum serum concentration and a greater area under the curve, at the same dosage as the prior art formulations in an accepted animal model. The novel formulations thus exhibit increased bioavailability allowing lower dosing to achieve an effective concentration.

All of the systems, compositions and/or methods disclosed and claimed herein can be made and executed without undue experimentation in light of the present disclosure. While the systems, compositions and methods of this disclosure have been described in terms of certain embodiments, it will be apparent to those of skill in the art that variations may be applied to the systems, compositions and/or methods and in the steps or in the sequence of steps of the method described herein without departing from the concept, spirit and scope of the disclosure. More specifically, it will be apparent that certain physical structures may be substituted for the physical structures described herein and the same or similar results would be achieved. All such similar substitutes and modifications apparent to those skilled in the art are deemed to be within the spirit, scope and concept of the disclosure as defined by the appended claims.

All documents, publications, patents, books, manuals, articles, papers, abstracts, posters and other materials referenced herein are expressly incorporated herein by reference in their entireties.

### REFERENCES

The following references, to the extent that they provide exemplary procedural or other details supplementary to those set forth herein, are specifically incorporated herein by reference.
Abouhamad W M, Manson M D. 1994. The dipeptide permease of Escherichia coli closely resembles other bacterial transport systems and shows growth-phase-dependent expression. Mol Microbiol 14 (5):1077-1092.
Abouhamad W N, Manson M, Gibson M M, Higgins C F. 1991. Peptide transport and chemotaxis in Escherichia coli and Salmonella typhimurium: Characterization of the dipeptide permease (Dpp) and the dipeptide-binding protein. Mol Microbiol 5 (5):1035-1047.
Calabresi L, Vecchio G, Longhi R, Gianazza E, Palm G, Wadensten H, Hammarstrom A, Olsson A, Karlstrom A, Sejlitz T, Ageland H, Sirtori C R, Franceschini G. 1994. Molecular characterization of native and recombinant apolipoprotein A-I Milano dimer. J Biol Chem 269:32168-32174.
Chaudhuri R R, Kahn A M, Pallen M J. 2004. coliBASE: An online database for Escherichia coli, Shigella and Salmonella comparative genomics. Nucleic Acids Res 32:D296-D299. Website: http://colibase.bham.ac.uk
Doeven M K, Abele R, Tampe R, Poolman B. 2004. The binding specificity of OppA determines the selectivity of the oligopeptide ATP-binding cassette transporter. J Biol Chem 279 (31):32301-32307.
Donovan J M, Benedek G B, Carey M C. 1987. Self-association of human apolipoproteins A-I and A-II and interactions of apolipoprotein A-I with bile salts: Quasi-elastic light scattering studies. Biochem 26:8116-8125.
Duplay P, Bedouelle H, Fowler A, Zabin I, Saurin W, Hofnung M. 1984. Sequences of the malE gene and of its product, the maltose-binding protein of Escherichia coli K12. J Biol Chem 259:10606-10613.
Gay, P., Le Coq, D., Steinmetz, M., Berkelman, T., Kado, C. I. 1985. Positive selection procedure for entrapment of insertion sequence elements in gram-negative bacteria. J. Bacteriol. 164: 918-921.
Grodberg J, Lundrigan M D, Toledo D L, Mangel W F, Dunn J J. 1988. Complete nucleotide sequence and deduced amino acid sequence of the ompT gene of Escherichia coli K-12. Nucleic Acids Res 16:1209-1209.
Gualandri V, Franceschini G, Sirtori S R. 1985. Identification of the complete kindred and evidence of a dominant genetic transmission. Am J Hum Genet. 37:1083-1097.
Guyer C A, Morgan D G, Osheroff N, Staros J V. 1985. Purification and characterization of a periplasmic oligopeptide binding proteins from Escherichia coli. J Biol Chem 260:10812-10818.
Hamilton C M, Aldea M, Washburn B K, Babitzke P, Kushner, S R. 1989. New method for generating deletions and gene replacements in Escherichia coli. J. Bacteriol. 171:4617-4622.
Hiles I D, Higgins C F. 1986. Peptide uptake by Salmonella typhimurium: The periplasmic oligopeptide-binding protein. Eur J Biochem 158:561-567.
Ho S V, McLaughlin J K, Thomas K E, Suda E J, Herberg J T, Dufield R L, Hunter A K. 2009. Reaction kinetics and optimization of the coppercatalyzed oxidation of ApoA-1M. Chem Eng Sci 64:2623-2630.
Hunter A K, Suda E J, Das T K, Shell R E, Herberg J T, Ramasubramanyan N, Gustafson M E, Ho S V. 2007. Impact of denaturation with urea on recombinant ApoA-1M ion-exchange adsorption: Equilibrium uptake behavior and protein mass transfer kinetics. Biotechnol J 2:110-120.
Hunter A K, Suda E J, Herberg J T, Thomas K E, Shell R E, Gustafson M E, Ho S V. 2008a. Separation of recombinant apolipoprotein A-I Milano modified forms and aggregates in an industrial ion-exchange chromatography unit operation. J Chromatogr A 1204:42-47.
Hunter A K, Wang X, Suda E J, Herberg J T, Shell R E, Thomas K E, Dufield R L, Gustafson M E, Mozier N M, Ho S V. 2008b. Separation of product associating E. coli host cell proteins OppA and DppA from recombinant apolipoprotein A-I milano in an industrial HIC unit operation. Biotechnol Prog 25:446-453.
Kramer R A, Zandwijken D, Egmond M R, Dekker N. 2000. In vitro folding, purification and characterization of Escherichia coli outer membrane protease ompT. Eur J Biochem 267:885-893.
Link A J, Phillips D, Church G M. 1997. Methods for generating precise deletions and insertion s in the genome of wild-type Escherichia coli: Application to open reading frame characterization. Bacteriol 179(20):6228-6237. PMID: 9335267.
McCarter J D, Stephen D, Shoemaker K, Rosenberg S, Kirsch J F, Georgiou G. 2004. Substrate specificity of the Escherichia coli outer membrane protease OmpT. J Bacteriol 18617:5919-5925.
Nissen S E, Tsunoda T, Tuzcu E M, Schoenhagen P, Cooper C J, Yasin M, Eaton G M, Lauer M A, Sheldon W S, Grines C L, Halpern S, Crowe T, Blankenship J C, Kerensky R. 2003. Effect of Recombinant ApoA-I Milano on coronary atherosclerosis in patients with acute coronary syndromes. JAMA 290(17):2292-2300.
Olson E R, Dunyak D S, Jurss L M, Poorman R A. 1991. Identification and characterization of dppA, an Escherichia coli gene encoding a periplasmic di-peptide transport protein. J Bacteriol 173 (1):234-244.
Sirtori C R, Calabresi L, Franceschini G, Baldassarre D, Amato M, Johansson J, Salvetti M, Monteduro C, Zulli R, Muiesan M L, Agabiti-Rosei E. 2001. Cardiovascular status of carriers of the apolipoprotein A-I (Milano) mutant: The Limone sul Garda Study. Circulation 103 (15):1949-1954.
Spurlino J C, Lu G-Y, Quiocho F A. 1991. The 2.3-A resolution structure of the maltose- or maltodextrin-binding protein, a primary receptor of bacterial active transport and chemotaxis. J Biol Chem 266:5202-5219.
Tame J R H, Murshudov G N, Dodson E J, Neil T K, Dodson G G, Higgins C F, Wilkinson A J. 1994. The structural basis of sequence-independent peptide binding by OppA protein. Science 264:1578-1581.
Vitello L B., Scanu A M, 1976. Studies on human serum high density lipoproteins. J Biol Chem 251:1131-1136.

What is claimed is:

1. An E. coli cell line modified to prevent expression of one or more of the oppA gene, the dppA gene, the malE gene and the ompT gene, or any combination thereof.

2. The cell line of claim 1, wherein the cell line comprises one or more chromosomal deletions that prevent expression of the one or more genes, or combination thereof.

3. The cell line of claim 2, comprising a deletion in the oppA gene, the dppA gene or the malE gene.

4. The cell line of claim 2, comprising a deletion in the oppA gene and the dppA gene.

5. The cell line of claim 2, comprising a deletion in the oppA gene, the dppA gene and the malE gene.

6. The cell line of claim 5, further comprising a deletion in the ompT gene.

7. The cell line of claim 2, comprising a deletion in the oppA gene, the dppA gene, the malE gene, and the ompT gene.

8. The cell line of claim 1, wherein the cell line produces a human apolipoprotein A-1.

9. The cell line of claim 8, wherein the cell line produces human apolipoprotein A-1 Milano.

10. The cell line of claim 8, wherein the apolipoprotein A-1 is produce through expression of a plasmid encoding the protein.

11. A method of producing recombinant apolipoprotein A-1 Milano comprising culturing the cell line of claim 9 under conditions that allow production of the protein; and collecting the recombinant protein from the cell culture media.

12. The method of claim 11, wherein the cell line comprises a deletion in the oppA gene, the dppA gene or the malE gene.

13. The method of claim 11, wherein the cell line comprises a deletion in the oppA gene and the dppA gene.

14. The method of claim 11, wherein the cell line comprises a deletion in the oppA gene, the dppA gene and the malE gene.

15. The method of claim 14, wherein the cell line further comprises a deletion in the ompT gene.

16. The method of claim 11, wherein the cell line comprises a deletion in the oppA gene, the dppA gene, the malE gene, and the ompT gene.

17. The method of claim 11, wherein the recombinant protein is purified through the steps of:
heat extraction of the protein-containing fraction of the cell culture media;
reduction of the protein-containing fraction by treatment with a thiol reductant;
contacting the reduced protein-containing fraction with a reversed phase capture column;
contacting the protein-containing fraction with an anion exchange column;
contacting the protein-containing fraction with a hydrophobic interaction phenyl column;
contacting the protein-containing fraction with a Cu(II) oxidant;
contacting the protein-containing fraction with an anion exchange Q column.

18. The method of claim 17, wherein the purified recombinant human apolipoprotein A-1 Milano contains less than 10 ng of host cell proteins per mg of protein.

19. An E. coli cell line that produces human apolipoprotein A-1 Milano and that is modified to prevent expression of each of the oppA gene, the dppA gene, the malE gene and the ompT gene.

20. An E. coli cell line that produces human apolipoprotein A-1 Milano and that has chromosomal deletions that prevent expression of each of the oppA gene, the dppA gene, the malE gene and the ompT gene.

21. The cell line of claim 19, wherein the human apolipoprotein A-1 Milano is produced through expression of a plasmid encoding the protein.

22. The cell line of claim 20, wherein the human apolipoprotein A-1 Milano is produced through expression of a plasmid encoding the protein.

23. A method of producing recombinant apolipoprotein A-1 Milano comprising culturing the cell line of claim 19 under conditions that allow production of the protein; and collecting the recombinant protein from the cell culture media.

24. A method of producing recombinant apolipoprotein A-1 Milano comprising culturing the cell line of claim 20 under conditions that allow production of the protein; and collecting the recombinant protein from the cell culture media.

25. A method of producing recombinant apolipoprotein A-1 Milano comprising culturing the cell line of claim 21 under conditions that allow production of the protein; and collecting the recombinant protein from the cell culture media.

26. A method of producing recombinant apolipoprotein A-1 Milano comprising culturing the cell line of claim 22 under conditions that allow production of the protein; and collecting the recombinant protein from the cell culture media.
